(19) United States
(12) Patent Application Publication
(10) Pub. No.: US 2006/0286606 A1
(43) Pub. Date: Dec. 21, 2006

(54) Clandestine Laboratory (Clan-Lab) Home Test Kit system, protocol, method and apparatus

(76) Inventor: Troy Lee Oliver, Madisonville, KY (US)

Correspondence Address: Troy Oliver, 330 Littlefoot Lane, Madisonville, KY, 42431, US

(21) Appl. No.: 11/453,027

(22) Filed: Jun. 15, 2006

Related U.S. Application Data
(60) Provisional application No. 60/690,953, filed on Jun. 17, 2005.

(51) Int. Cl. G01N 33/53; G01N 33/00

(52) U.S. Cl. 435/7.1; 436/96; 436/97

(57) ABSTRACT

This invention is comprised of a system, protocol, method and apparatus for the assessment of properties that may have been, or are being, subject to clandestine drug manufacturing and/or processing activities. The invention includes a comprehensive home test kit to be used in or upon a suspect premises to detect, identify, and delineate toxic chemical hazards that may have originated from an illegal drug making operation. The test kit is designed to be conveniently equipped with all-inclusive content consisting of an assortment of user-selected sampling equipment, media, containers, materials, documentation, instruction manual, as well as an audio-visual instructional media pack. This Kit is designed to enable a person of average intelligence to conduct the sampling activities and the Kit to a designated analytical laboratory for processing and reporting of results in order to determine the risk presented by a property and damages it may have sustained.

### PRIORITY CLAIM

The present application claims priority from U.S. Provisional Application No. 60/690,953 filed Jun. 17, 2005, entitled “Clan-Lab Home Test Kit—A novel concept and method for sampling and analyzing suspect properties to determine qualitative and quantitative evidence of chemical hazards pursuant to suspected, or known, former illegal drug manufacturing activities.”

### BACKGROUND OF THE INVENTION

Situation

Although, great progress has been made during recent years in the battle against most aspects of the illegal narcotics trade, clandestine drug manufacturing and processing activities are now publicly recognized as portions of our national crime problem that continue to expand despite law enforcement's efforts. In fact, the problem presented by these illegal clandestine drug laboratories may be the fastest growing segment of crime in America today; and undisputedly, it has negatively affected and endangered many people . . . and many others are suffering harm even now who may have no idea of the dangers awaiting them within their own home.

The crime of clandestine drug manufacturing and processing has primarily developed over the last three decades. Over the last decade, the development of the internet has dramatically increased the development and distribution of the illegal, “do-it-yourself” drug manufacturing enterprise by disseminating drug manufacturing technology and made available a variety of narcotics chemistry recipes to the general public.

Methamphetamine (or meth) labs are one such type of clandestine laboratory operation that has grown rapidly during the last decade in part because of the internet's influence and the fact that demand for such drugs has continued to rise as well. However, the demand for meth has not been met by a few large clandestine labs, as was the case in the previous decades, but rather over the last decade, this demand has been satisfied by many clandestine labs dispersed across the nation in both rural and metropolitan areas alike. This clandestine drug manufacturing trend is most certain to continue its rapid rate of growth as more demand for illegal narcotics and more criminal ingenuity combine to create new dangers and problems for our nation.

When an illegal drug manufacturing operation, or clandestine drug laboratory, is seized by law enforcement all containerized drugs and chemical substances, discovered at the crime scene, are taken by the authorities. Generally, whatever chemicals may have spilled, absorbed, or have otherwise been released, are simply left behind. This residual contamination issue is a serious unchecked problem from both a human health and safety standpoint as well as from an environmental perspective.

In most scenarios where a clandestine drug lab is being operated, the suspect lab operator is not the owner of the property where the crime is being committed. Illegal drug labs are typically located on properties that are owned by others and are either being rented or trespassed upon by the perpetrator. In the vast majority of these situations, the crime scene property is chemically contaminated to some degree by the clandestine drug manufacturing process and is not properly remediated. Therefore, the unabated contamination issue remains and continues to present a danger to the safety and health of future occupants and to the community.⁷

There are two affected groups of individuals within the class of victims, which are most severely impacted by this problem:
1^{st} Group This group consists of the innocent tenants of properties formerly used for the manufacture of illegal drugs. This innocent third-party (I3P) victims group includes those persons (i.e.: residents, workers, visitors, playing children, etc.) who may have been exposed to chemical hazards in or upon the crime scene property.
2^{nd} Group This group consists of the innocent property owner of the clandestine drug laboratory crime scene property.

It is anticipated that these affected groups will either be the end user or customer to this invention or be the object of a service or benefit by a customer or end user to this Kit.

Unique Dangers

It is widely known that many of the individual chemicals typically involved in clandestine methamphetamine manufacturing process not only cause cancer, but also lead to other serious health effects and/or birth defects.⁶ These carcinogens and toxic mixtures, over time, impact those who have been chronically exposed; and the latency period of many such exposure related illnesses may not actually manifest themselves into symptoms that are easily medically detectable for years to come. Then once revealed, the innocent victim is seriously endangered because the damage has advanced into a more progressive, life threatening disorder.

Despite multiple warnings from a variety of Government agencies, drug lab crime scenes throughout the majority of the United States are most often neither restricted nor monitored after the point of seizure. Generally, property owners at their own discretion are left to fend for themselves and determine the necessity and extent of whatever, if any, cleanup activities are to be conducted. Many times, new tenants are moved in as soon as possible with little or no cleanup activity taking place, whatsoever.

Today, one of the most publicly provoking aspects of this problem is the debuting crisis of “Drug Endangered Children,” or DEC. In recent years, bio-monitoring studies have overwhelmingly concluded that innocent children are the most seriously impacted by the physical and chemical hazards associated with living in a residence that either is, or was, used as a clandestine drug laboratory. In fact, of the children tested thus far in these various studies, a substantial percentage have demonstrated elevated levels of toxic substances in their bloodstream.⁵ This development raises the priority of this problem to a crisis level.

Of those children tested, who have lived in homes containing clandestine drug laboratories, over 40% have demonstrated elevated levels of toxic substances in their bloodstream according to a California Bureau of Narcotic Enforcement research study.⁶ Other sources, such as the U.S. Dept. of Justice, have published similar findings, which also demonstrate a rising trend of blood borne toxins discovered in these Drug Endangered Children.⁷ The problem is indeed serious and warrants serious consideration on behalf of the many innocent victims involved.

In 2005, the EPA released new findings and proposed cancer risk management guidelines, which reveal strong evidence that changes its previously assumed position that cancer risks to children were no greater than to similarly exposed adults. In their newly published findings, the EPA has stated that “children two years old and younger are ten times more vulnerable than adults to certain chemicals and that children between the ages of two and sixteen are three times more vulnerable to certain chemicals.”²⁵

It is not an unlikely assumption to project that potentially today more children are being placed at risk of cancer due to the methamphetamine crisis than any other known environmental hazard within our nation. Karen P. Tandy, Administrator of the U.S. Drug Enforcement Administration was quoted in a National Jewish Medical and Research Center article as saying, “The high levels of toxins dispersed during meth manufacturing expose innocent and unwary citizens to poisons that can be silent killers. ”²⁶

U.S. Department of Justice's National Drug Intelligence Center recently released its annual report titled, “The National Drug Threat Assessment 2006” wherein it declares that the clandestine laboratory problem, “continues to jeopardize the safety of citizens, adversely affect the environment, and strain law enforcement resources. Children, law enforcement personnel, emergency responders, and those who live at or near methamphetamine production sites have been seriously injured or killed as a result of methamphetamine production. Chemical waste from methamphetamine laboratories has killed livestock, contaminated streams and soil, and destroyed vegetation.”¹

Unique Challenges

The problem is unique; in that, the chemicals associated with clandestine drug manufacturing vary widely from application to application due to the illegal nature of the enterprise. Many chemicals are quite volatile and have odors that are offensive enough to warn occupants of the presence of a hazard. Other chemicals may be present in these situations, which continually release vapors into the indoor air at or below olfactory threshold levels whereas a normal person's sense of smell will not be sensitive enough to warn them of the potential dangers.

It is widely known that many of the individual chemicals typically involved in clandestine methamphetamine manufacturing process cause cancer and other serious health effects as well as birth defects. The nature of this crime is also such that many different kinds of chemicals are combined into an infinite variety of mixtures with a wide range of toxic exposure consequences that are impossible to accurately predict and many are very difficult to detect and identify using standard toxic chemical monitoring protocol.

Need for This Invention

The issue of toxic hazards facing innocent citizens nationally due to the illegal past drug manufacturing acts of others, is a complex, highly variable, ever-changing puzzle of obstacles that encompasses many problems in the areas of logistics, legalities, and economic constraints. It is a national problem without a solution. It has been a cause without a crusader.

This invention is a legitimate necessity to fulfill a national need and will make it possible for relief and remedy measures to reach those who are harmed by this crime. Furthermore, this Clandestine Lab Home Test Kit invention allows the customer or end user to decide how much accuracy and precision they can afford since they are, more often than not, forced to bear the burden of discovery. The concept offers customers a general screening option and either a piece of mind or a healthy concern for whatever said screening may have revealed.

In most situations, the average citizen is:
1. not able to understand the danger involved with meth chemical residues,
2. not able to quantify the nature and extent of the contamination problem,
3. not able to adequately address the cleanup of the toxic hazard, and they are
4. not able to present a qualified claim for victims' benefits, insurance coverage, or other forms of public or private assistance.

Obviously, the current tenants to these problem properties and the owners of these properties are at an incredible disadvantage. The unique nature of a danger that may or may not be easily detectible, and even more difficult to communicate and quantify, has rendered a great injustice onto the shoulders of these innocent victims.

Application Scenario

The particular arrangement of any given clandestine laboratory defies most attempts categorize and classify according to a standard; in as much as, the variability of the illegal drug making process and chemistry is as diverse as the human imagination. However, most operations include a “cook” process of some sort and therein lies the primary mechanism of contaminate transport within a structure. Mishandling of chemical related substances including spilling and/or dumping of such materials is the other predominant mode of contaminate distribution observed at clandestine laboratory sites. Accordingly, the Clandestine Lab Home Test Kit is designed as a tool for discovering contaminates related to the “cooking” and mishandling processes relative to a past or present clandestine narcotics manufacturing or processing activities.

The “cook” mechanism in a drug lab releases steams, aerosols, vapors, and gases into the atmosphere. Some of these chemicals substances precipitate or settle out as a film upon the surfaces of the structure as well as upon items of real and personal property. Other chemical substances are absorbed into the structural materials themselves as well as into items of both real and personal property.

The Clandestine Lab Home Test Kit, in its various configurations, is designed to answer a progression of customer or end user questions?
1) Is there evidence of clandestine narcotics manufacturing or processing activities?
2) What chemicals are present and how strong is the concentration of said chemicals?
3) Where are these chemical substances coming from and how much area and property or objects have been impacted or contaminated?

In its basic or Q1 configuration the Kit is designed to answer the first question by investigating surface deposit residues for narcotics related precipitates or films and also provides for air sampling to yield evidence of absorbed chemical substances that may be volatilizing or desorbing from the property itself or from objects within the structure. The other Kit configurations and variations thereof are designed to provide a means and method for more detailed investigation activities pursuant to the Kit's comprehensive assessment purposes.

### REFERENCES CITED

U.S. Patent Documents

| | | | |
|---|---|---|---|
| 7,060,505 | June 2006 | Guirguis | 436/514 |
| 5,994,144 | November 1999 | Nakajima, et al | 436/116 |
| 5,419,209 | May 1995 | Sepe | 73/863 |
| 4,840,912 | October 1988 | Glattstein | 436/92 |

Other Publications:
1) “National Drug Threat Assessment 2006.” National Drug Intelligence Center, U.S. Department of Justice. Product No. 2006-Q0317-001, Page 3, 37
2) “Review of Contaminant Levels: Guidelines for Clandestine Drug Lab Cleanup”, By Harriet Amman, Ph.D. Office of Environmental Health Assessments, September 2000.
3) “Meth-coated homes spur new push for Federal research (AP)” Article written by Devlin Barrett of The Associated Press, Feb. 16, 2005.
4) “Methamphetamine Situation: A Growing Domestic Threat.” Methamphetamine Situation in the United States, U.S. Dept of Justice, Drug Enforcement Administration. http://www.fas.org/irp/agency/doj/dea/product/meth/threat.htm
5) “Children in Clandestine Laboratories: the California Experience” paper presented May 29, 1997 by Thomas J. Gorman, California Bureau of Narcotic Enforcement at the National Methamphetamine Drug Conference, Omaha, Nebr.
6) “Children at Risk.” U.S. Dept. of Justice, National Drug Intelligence Center, Information Bulletin, July 2002. Publication No. 2002-LO424-001.
7) “Children at Clandestine Methamphetamine Labs: Helping Meth's Youngest Victims” by Karen Swetlow, U.S. Dept. of Justice, Office for Victims of Crime, OVC Bulletin. June 2003. Pg. 6.
8) Testimony presented before the U.S. House of Representatives Committee on Government Reform, Subcommittee on Criminal Justice, Drug Policy and Human Resources, Jun. 28, 2004, by Shirley Louie, M. S., CIH, Chief Environmental Epidemiologist, Arkansas Department of Health, Little Rock, Ark.
9) “Multi-Agency Partnerships: Linking Drugs with Child Endangerment,” Governor's Office of Criminal Justice Planning (OCJP), May 12, 2003. Sacramento, Calif., p. 9.
10) Testimony of James MacDonald, U.S. E.P.A. On-Scene Coordinator before the U.S. House of Representatives Committee on Government Reform, Subcommittee on Criminal Justice, Drug Policy and Human Resources Committee on Government Reform, Jun. 28, 2004.
11) “Unauthorized Production of Methamphetamine: Legal Responsibilities of Land and Rental Property Ownership” Written by William H. Fortune, May, 2004. Professor University of Kentucky, College of Law. Publication HEEL-HEH.206. http://www.ca.uky.edu/fcs/FACTSHTS/HEEL-HEH.206.pdf
12) “Dangers of Exposure to Chemicals Used in Meth Labs,” Written by Holly E. Hopper, M. R. C. Extension Associate for Health, October, 2004. University of Kentucky, College of Agriculture, Publication HEEL-HEH.205. http://www.ca.uky.edu/fcs/FACTSHTS/HEEL-HEH.205.pdf
13) “Protecting First-Responders from Clandestine Meth Labs.” Georgia Institute of Technology. May 10, 2005. http://www.newswise.com/articles/view/511687/
14) “Walk Your Land: The Extension Agent's Guide For Protection of Private Property Against Unauthorized Clandestine Methamphetamine Production.” Published by the University of Kentucky, College of Agriculture Health Education through Extension Leadership, HEEL Program. Page9.
15) “Ordering Restitution to the Crime Victim”, USDOJ OVC Legal Bulletin #6. November 2002.
16) “If You Don't Have a Dime, Who Pays for the Crime?—The Mandatory Victims Restitution Act” Written by Kristin I. Tolvstad. United States Attorneys' Bulletin. Victims Rights, January 1999, Volume 47, Number 1. Pages 13-14.
17) “The Attorney General Guidelines for Victim and Witness Assistance: United States Attorneys' Offices' Responsibilities to Victims” by Camille Bennett, United States Attorneys' Bulletin. Victim-Witness Issues II, March 2003, Volume 51, Number 2. Pages 2-7.
18) “Restitution Update” Written by Catharine M. Goodwin. United States Attorneys' Bulletin. Victim-Witness Issues, January 2003, Volume 51, Number 1. Pages 13-19.
19) “Overview.” DEA's Victim Witness Assistance Program. DEA Website Resource. http://www.dea.gov/resources/victims crime.html
20) “National Clandestine Laboratory Seizure Report—Form EPIC 143 (06-2004).” U.S. Drug Enforcement Administration, EPIC Group. OMB No. 1117-0042.
21) “Attorney General Guidelines for Victim and Witness Assistance,” 2000 Edition, Section F. Page 10.
22) “Asset Forfeiture Policy Manual.” U.S. Dept. of Justice, Criminal Division, July 1996.
23) “Exposure risk higher for kids: EPA worries about carcinogens” by John Heilprin, Associated Press. Mar. 31, 2005.
24) “Attachment to the Attorney General Guidelines for Victim and Witness Assistance,” 2000 Edition. U.S. Department of Justice Policy Document. http://www.usdoj.gov/ag/readingroom/2000victim2.htm
25) Smiths Detection/Barringer Instruments Website. IONSCAN® 400B Product Information Section. http://194.105.117.18/products/Default.asp?Product=16
26) “Toxic Brew of Chemicals Cooked Up in Methamphetamine Laboratories.” National Jewish Medical and Research Center, Jan. 19, 2004. http://www.nic.org/news/meth results.html

### DISCUSSION OF PRIOR ART

Currently, there are no comprehensive screening or measurement technologies available for the average citizen to assess the impact of this particular type of hazard presented by illegal drug manufacturing activities. In fact for this unique application, there are also no comprehensive screening kits available to professional investigators in the law enforcement or public health fields. The Clan-Lab Home Test Kit invention offers a system, a protocol, a method, and an apparatus collectively designed to address this need and national problem with an innovative solution.

Present EPA recommended analytical criteria for the identification of unknown chemical substances in both ambient air concentrations and surface residues represents the ideal, this Clandestine Lab Home Test Kit invention is designed to satisfy the “real” and provide an economical alternative to current industrial hygiene methods that may be applied toward the evaluation of such properties. In reality, both the price and complexity of the “one size fits all”, or ideal, analysis have effectively separated an estimated quarter of a million innocent citizens from any analysis relief at all and the associated hazards of this national scale problem have continued to impact and harm many innocent citizens for over a decade now. The Clandestine Lab Home Test Kit is a fresh attempt to establish a new technological invention and, in doing so, lay the foundation for a new field of science related to the identification and abatement of the nationally dispersed and escalating problem caused by illegal drug manufacturing acts.

This invention differs from all prior art environmental investigative techniques; in that, this invention has various quantitative and qualitative components with a designed flexibility incorporated into the process. Additionally, this invention, by design, benefits from perpetual research toward the analysis of subject properties and will identify local, regional, and nation trends in clandestine manufacturing and processing chemistry.

The only so-called drug lab home test kits being promoted today are based upon a calorimetric indicator principle; whereas, a reagent solution is applied to a sample of the suspected narcotic substance and a color change occurs from the chemical reactions thereof. The single or multiple reagent kits are effective and useful as a field expedient method for qualification determination of raw narcotics substances, but have limited utility in identifying residual narcotics substances in trace amounts that may still be harmful to human life and health. Additionally, these reagent only kits are not effective standalone devices to be used for ascertaining the airborne contamination levels and surface chemical deposits typically associated with past or present illegal clandestine drug manufacturing or processing activities.

This invention does not rely upon calorimetric indicator solutions to identify narcotic substances; rather this invention only includes calorimetric tests at a customer or end user's request to help identify suspicious solid or liquid substances. The calorimetric indicator tests rely upon having a substantial portion of narcotic's residue in order to facilitate the desired reaction. Trace quantities of narcotic substances, such as a methamphetamine residue left upon walls and other interior dwelling surfaces after an illegal drug manufacturing act has taken place, do not allow for identification via the colorimetric indicator process. Conversely, Ion Mobility Spectrometry (IMS) technology, Ion Track technology and Gas Chromatograph Surface Ionization Detector (GC-SID) are capable of detecting residues in the picogram to nanogram range.

Currently Smiths Detection (http://trace.smithsdetection.com) manufactures an IMS IonScan Technology based product known as the Sabre 4000. Likewise, Scintrex Trace Corporation (http://www.scintrextrace.com/) manufactures another portable narcotics detection device known as the N2000, which operates off the GC-SID technology. Each of these devices is portable and was developed for drug interdiction and law enforcement purposes to detect minute quantities of narcotics substances. These devices are advantageous for giving the user an almost instantaneous reading at the scene of the investigation effort. Unfortunately, these devices are expensive and are relatively rare in commercial application outside of official government use. Additionally, these portable devices are subject to error and malfunction when taken into contaminated atmospheres such as those that may be presented by an illegal drug lab operation.

The Phase Zero Environmental Assessment (U.S. Pat. No. 5,419,209) consisted of a system an protocol designed for the environmental assessment of residential properties; yet the patent contains no mention of evaluating properties for past or present evidence or impact associated with drug manufacturing or processing activities upon residential properties.

Additionally, the Phase Zero kit was prepared for use by “environmental home inspectors” using EPA testing methodology and conventional industrial hygiene apparatus. Conversely, the Clan-Lab Home Test Kit was designed for use by normal citizens of average intelligence; whereas, the training needed to conduct said test does not come from special schools or classes, but by audio-visual training media included in said Kit. Furthermore, the Clan-Lab Home Test Kit is not limited to established sampling methodology or equipment; rather, said Kit was specially prepared to comprehensively provide all training, equipment, materials, and documentation necessary for performing a regimen of sampling activities unique to this application

### SUMMARY OF THE INVENTION

This invention consists of an all-inclusive kit comprised of individual components necessary to evaluate gaseous, liquid, and solid substance residues relative to determining the nature and extent of chemical contamination of subject properties with suspected or known histories of clandestine manufacturing and/or processing of illegal narcotic and toxic substances.

This invention also includes a system and protocol for a comprehensive home test kit to be used to detect, identify, and delineate toxic chemical hazards associated with illegal clandestine drug manufacturing or processing activities.

This Clandestine Lab Home Test Kit invention is the first self-test concept kit of its kind designed to comprehensively evaluate the toxic hazards relative to the residues of illegal drug manufacturing acts. This approach represents a novel technological concept; whereas, regular private citizens are provided within the kit both the training and resources to conduct the tests themselves. This invention further provides a method for giving customers the option of being able to specify the content of custom configured kits via a menu of testing components and costs. Conversely, ambient air sampling and surface wipe tests have been historically conducted exclusively by trained health and safety professionals, industrial hygiene personnel, or other such environmental technicians, all of whom have received specialty training and have been equipped for this manner of on-site sampling and analysis.

This invention extensively utilizes audio-visual media to instruct private citizens as to how to perform the necessary Clandestine Lab Home Test Kit tests. Accordingly, these private citizens are also instructed as to how they can properly return said kit for analysis to a laboratory, which is specially equipped and configured for this manner of analysis. The training formats for this audio visual component will include standard instructional media presentations in both DVD and VHS formats as well as on-line web based video media formats such as presentations prepared using Quicktime, Windows Media, and other such audio visual electronic on-line video presentation formats. Additionally, printed instructional booklets will also be included within the kit configuration for procedural reference and information reinforcement.

### OBJECTS OF THE INVENTION

It is an object of this invention to provide a system, protocol, method and apparatus for a test kit to be used to identify and/or quantify chemical substances pursuant to the illegal manufacture of controlled narcotic substances as specifically defined by the schedules of controlled substances known as Schedules I, II, III, IV, and V as identified in 21 USC Sec. 812 (TITLE 21—FOOD AND DRUGS CHAPTER 13—DRUG ABUSE PREVENTION AND CONTROL SUBCHAPTER I—CONTROL AND ENFORCEMENT Part B—Authority To Control; Standards and Schedules), and/or a variety of organic and inorganic chemical ingredients, precursors and recursors associated with illegal controlled substance manufacturing or processing activities.

It is another object of this invention to provide a system, protocol and method for incorporating an audio-visual media training component into said Clan-lab Home Test Kit to safely enable average citizens to conduct the various tests and return the kit to a qualified laboratory facility, equipped to analyze the components of said Test Kit and provide a report of the findings thereof.

It is yet another object of this invention to provide a system, protocol, method and apparatus for enabling law enforcement officers, child protection workers, public health officials, medial personnel, representatives of the court systems, home inspectors, health and safety officials, environmental officials, municipal workers, park rangers, academic and research personnel, and other government or military personnel to safely test suspect premises or properties to identify, distinguish, and measure evidence of chemical hazards pursuant to suspected, or known, former illegal drug manufacturing and/or processing activities.

It is yet another object of this invention to provide a system, protocol, method and apparatus for cost effective environmental and safety hazard screening of real estate properties for impact and damage relative to illegal drug manufacturing and/or processing activities and to provide assurances to those individuals who either are currently, or plan to be, in contact with said suspect properties.

It is yet another object of this invention to provide a system, protocol, method and apparatus to assist buyers of real estate property in determining whether or not said properties have been subject to impact and damage relative to illegal drug manufacturing and/or processing activities and as such to provide a mechanism for ascertaining the damages to said properties as well as the cost for the remediation of the toxic contaminants that may have impacted said properties.

It is yet another object of this invention to provide a method and apparatus for collecting contaminates from indoor atmospheres suspected of past or present illegal drug manufacturing and/or processing activities and provide for the extraction of said contaminates through a thermal desorption process and provide for the qualitative and/or quantitative analysis of the same.

### BRIEF DESCRIPTION OF DRAWINGS

FIG. 1 is an artistic rendering showing the contents of the Clandestine Laboratory (or Clan-Lab) Home Test Kit in its most basic qualification (or Q1) configuration;

FIGS. 2A-2F (6 charts) are a flowchart diagram, which describes the steps taken by the Clan-Lab Home Test Kit user, who is conducting the sampling and assessment activity pursuant to the nature of the Kit.

FIG. 3 is an apparatus drawing showing two views of the Vapotrap Air Toxics Sample Canister Assembly. The cutaway view shows a profile of said Canister Assembly and identifies the components of this particular embodiment. The perspective view shows how the Canister Assembly's primary external components are arranged in its operational form.

FIG. 4 is a process drawing showing Vapotrap Sample Processing Method 1, which relates to a means of taking a Vapotrap capsule and/or subpod component and subjecting said capsule or component to a thermal source of heat and/or steam; whereas the chemical components trapped therein are extracted or released by said thermal forces and are routed by the system into a vapor expansion and mixing chamber either directly or through a chilling unit. This drawing is identified into three major operational segments or units of sample preparation comprising: (1) the extraction unit, (2) the chiller unit, and (3) the vapor measurement unit.

FIG. 5 is a process drawing showing Vapotrap Sample Processing Method 2, which relates to a means of taking a Vapotrap Canister Assembly, which contains the Vapotrap capsule and subpods, and subjecting said Canister to a thermal source of heat and/or steam; whereas the chemical components trapped therein are extracted or released by said thermal forces and are routed by the system into a vapor expansion and mixing chamber either directly or through a chilling unit. This drawing is identified into three major operational segments or units of sample preparation comprising: (1) the extraction unit, (2) the chiller unit, and (3) the vapor measurement unit.

FIG. 6 is a process drawing showing the final step of the Vapotrap Air Toxics Sample Analytical Testing Process, which is a continuance of the processes identified in FIG. 4 and FIG. 5. This drawing identifies the fourth major operational segment or unit of sample preparation and processing activity or (4) the Analytical Unit; whereas, said unit graphically presents a number of analytical measurement mechanisms to be employed given the known or suspected chemistry of the potential clandestine laboratory operation being investigated.

### DETAILED DESCRIPTION OF THE INVENTION

Clandestine Laboratory (or Clan-Lab) Home Test

While this invention is satisfied by embodiments in many different forms, there is shown in the drawings and will herein be described in detail, preferred embodiments of the invention with the understanding that the present disclosure is to be considered exemplary of the invention and is not intended to limit the invention to the embodiments illustrated. The scope of the invention will be measured by the appended claims and their equivalents. Additionally, this invention offers other objects and many advantages as will be readily appreciated as said invention becomes better understood by reference to the following description:

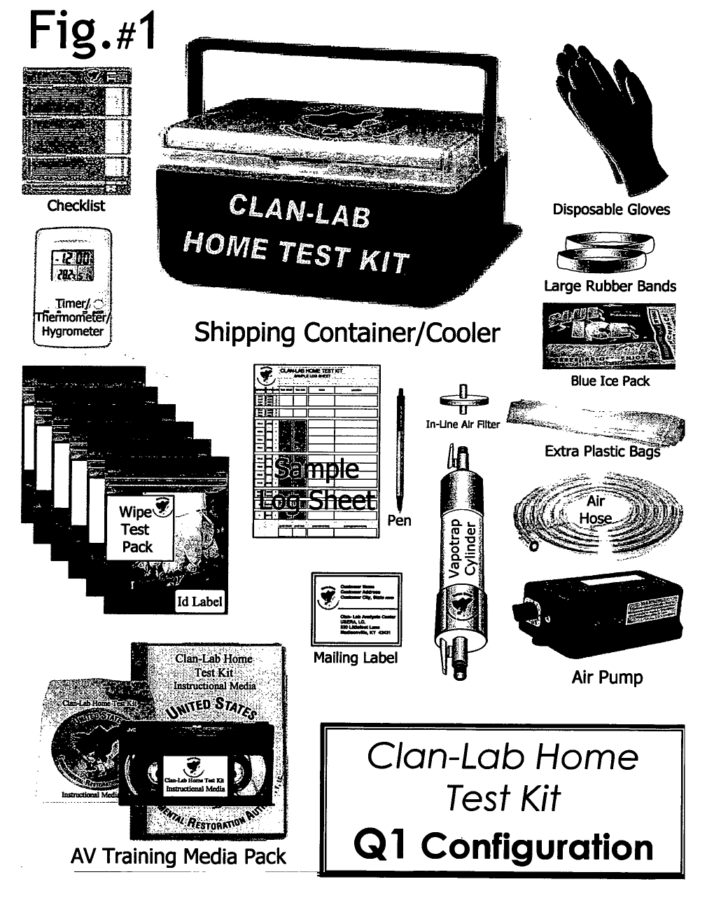

The first embodiment of this invention is the Clandestine Laboratory (or Clan-Lab) Home Test Kit, which is comprised by the assortment of components contained therein. FIG. 1 is an illustration, which shows the contents of the Clandestine Laboratory (or Clan-Lab) Home Test Kit in its most basic qualification (or Q1) configuration. The actual number and type of components selected for the Kit will be dictated by the end user or customer, giving respect to the anticipated or known risks to the property, which will be subject to the application and use of said Test as well as the customer's ability to afford investigative assurances.

The three major Kit configurations are qualitative (or Q1), quantitative (or Q2) and combination (or Q3). Table 1 outlines the general list of available analytes per given general Kit configuration and an inventory arrangement is included for each said Kit configuration. Typically, an initial property screening will be a more qualitative nature and follow-up testing, if necessary, will include a second battery of testing to be performed upon said subject premises employing either a Test Kit in the quantitative (or Q2) and combination (or Q3) configuration. The availably of information or knowledge of the site suggesting that it was indeed used for clandestine drug manufacturing or processing activities, and/or in the case that distinct evidence of chemical contamination upon said premises is obvious, the end user or customer of said Kit may elect to custom configure a Q3 or combination Kit to expedite the investigation activities to be conducted pursuant to the Kit's purpose.

In a basic format or Q1 configuration (FIG. 1), the Clan-Lab Home Test Kit is intended to investigate several surface deposit samples as well as evaluate indoor air quality for evidence absorbed, spilled, and/or released chemical substances consistent with illegal drug manufacturing and/or processing activities. Even though some degree of chemical identity and quantity information may be developed in the testing process, the objective of the Q1 or qualitative test is to answer the question as to whether or not a clandestine laboratory has impacted the premises. Rather, the quantitative or Q2 test kit configuration is specifically ordered to both identity and quantify the chemical substances present at the site being investigated.

In the Q1 Kit configuration, the purpose of said Kit is to provide the means, methods, and tools for investigating surface deposit residues for narcotics related precipitates or films and also provides for air sampling to yield evidence of absorbed chemical substances that may be volatilizing or desorbing from the property itself or from objects within the structure. The other Kit configurations and variations thereof are designed to provide the means, methods, and tools more detailed investigation activities pursuant to the Kit's comprehensive assessment purposes.

When circumstances dictate, a combination kit or Q3 configuration can be custom assembled by the Kit provider giving consideration for the user's particular needs and budget. There are many unique embodiments and possible combinations present in the Q3 Kit arrangement. In this manner, a very custom solution can be tailored to the unique challenge of the individual application as opposed to the “one size fits all” approach that may not be practical or affordable to the user.

Table 1 is a summary of Kit configuration inventories and lists a number of elements that can be chosen by a Kit user to qualify, quantify, fingerprint, delineate, and estimate contamination impact due to the alleged influence of clandestine drug manufacturing or processing activities.

TABLE 1
Clandestine Laboratory (Clan-Lab) Home Test Kit
Inventory of Content per Configuration

| | | Kit | | | |
|---|---|---|---|---|---|
| | | Config- | | | |
| Index | | uration | | | |
| # | Item | Q1 | Q2 | Q3 | Return? |
| 1 | Container/Cooler | X | X | X | X |
| 2 | Instruction Manual | X | X | X | |
| 3 | AV Instructional Media Pack | X | X | X | |
| 4 | Sampling Report & Checklist | X | X | X | X |
| 5 | Wipe Sample Packs (Drugs 1) | X | X | X | X |
| 6 | Vapotrap Cylinder | X | X | X | X |
| 7 | Air Pump, Hose, Fittings, & Inline Filter | X | X | X | X |
| 8 | Iodine Wipe Test Pack | X | X | X | X |
| 9 | Electronic Timer, Thermometer, | X | X | X | X |
| | & Hygrometer | | | | |
| 10 | Return Label Preprinted | X | X | X | X |
| 11 | InkPen | X | X | X | |
| 12 | Blue Ice Pack | X | X | X | X |
| 13 | Plastic Bag for wipe pack bags | X | X | X | X |
| 14 | Extra Latex Gloves | X | X | X | |
| 15 | Sample Swab Pack | | X | X | X |
| 16 | Wipe Sample Packs (Drugs 2) | | X | X | X |
| 17 | pH Test Strip Pack | | | X | |
| 18 | Distilled or Deionized Water flask | | | X | |
| 19 | Ammonia Wipe Test Pack | | | X | X |
| 20 | Phosphine Wipe Test Pack | | | X | X |
| 21 | Lead and Mercury Wipe Test Pack | | | X | X |
| 22 | Diffussive Media Sampling Pack - | | | X | X |
| | Badge, Wafer, Cartridge, and/or Tube | | | | |
| 23 | Suspect Materials Test Pack | | | X | X |
| 24 | Photoionization Meter Pack | | | X | X |
| 25 | Colorimetric Narcotics Reagent Pack | | | X | X |
| 26 | Colorimetric Test-Strip Pack for Narcotics | | | X | X |
| | Precursor & Recurser Substances. | | | | |
| 27 | Septic Tank sample pack | | | X | X |
| 28 | Soil Test sample pack | | | X | X |
| 29 | Well water sample pack | | | X | X |
| 30 | Surface water sample pack | | | X | X |
| 31 | Unknown Liquids sample pack | | | X | X |
| 32 | Unknown Solids sample pack | | | X | X |
| 33 | Colormetric Sample Kits | | | X | |
| | (Inorganic Compounds) | | | | |
| 34 | Toxicology Reference Primer | | | X | |
| 35 | Wipe Sample Packs (Other) | | | X | |
| 36 | Safety Glasses | | | X | |
| 37 | Chemical Gloves | | | X | |

The Self-Test Nature of a Comprehensive Home Test Kit

It is another unique embodiment of this invention that this is a home test kit in the sense that it was prepared not only for an industrial hygienist, an environmental specialist or even a health and safety expert, but that an average citizen would have the means of safely being able to sample a premises themselves thus making the benefits offered by this invention available, and affordable to a far greater populace.

By making extensive use of audio-visual media and illustrated documentation, the Clandestine Lab Home Test Kit will safely and efficiently instruct private citizens as to how they can perform the necessary sampling test activities. A key, and extremely valid concern is the health and safety of the Kit user. Obviously, there are situations where entering an area formerly or currently used as a clandestine lab operation may endanger a person's life and health. The instructional training media and documentation included in said Kit will strongly assert and reinforce the message that if danger signs are observed and/or expected the Kit user is to cease immediately all sampling efforts and contact appropriately trained professionals for assistance. However, it should also be noted that such instances are the rare exception and not the rule for clandestine laboratory operations. In fact, it is anticipated that many users of said Kit may actually already be residing or otherwise occupying a premises and have a valid concern for the safety of themselves or their family.

It is an assumption that potential Kit users have already visited the site subject to suspicion and thus desire to ascertain whether or not such premises are chemically contaminated and if so, to what degree is the damage distributed upon said premises and how can it be safely removed? The Clan-Lab Home Test Kit offers answers that most persons can afford. Conversely, typical “worst case assumptions” and standard industrial hygiene protocol have placed the price for answers and assistance beyond all, but the most affluent Americans. For this reason on a very tiny fraction of the more than one hundred thousand sites documented thus far in the DEA's Clandestine Laboratory Seizure System database have been assessed for hazards, not to mention the number of sites that are not reported by law enforcement officials into this record system, which many officials estimate only 1 of every 4 sites are actually reported because of the time it takes a law enforcement officer to fill out the four page EPIC/CLSS reporting form.²⁰ When the true scale of this problem is appraised, the potential victim distribution estimates are staggering. It is an urgent objective of this invention to place real relief in the grasp of those who are needlessly suffering because they can't afford the price of conventional scientific protocol.

As long as the Kit's instructions are followed and warnings are heeded, a Kit user or customer is placed in no greater degree of danger than that they would normally face by physically wandering around a suspect property observing sights and smells while they attempt to assess, clean, paint, or deodorize the suspected damages. The time actually required for a user to conduct the necessary sampling activities for the Kit is minimal because the air-monitoring portion of the Kit's testing protocol can operate on an unattended basis until the designated test period has been completed and at such time the Kit user can later return to the premises and retrieve the Kit's remaining articles.

Table 2 outlines the Audio-Visual (AV) Instructional Media Content embodiment of the Clandestine Laboratory (Clan-Lab) Home Test Kit; whereas, the Kit utilizes a recognized learning characteristic approach to effect the dissemination and retention of the desired information and communicate said information in an audio-visual context to the extent that a citizen, of average intelligence, can understand the purpose of the Kit and be able to perform the sampling procedure upon a premises subject to investigation.

TABLE 2
Clandestine Laboratory (Clan-Lab) Home Test Kit
Audio-Visual (AV) Instructional Media Content

| | |
|---|---|
| I. | Introduction |
| II. | Toxic Dangers from Clandestine Drug Manufacturing |
| III. | Personal Safety Precautions |
| IV. | The Clandestine Home Test Kit |

| | |
|---|---|
| A. | General Introduction to Concept and Configurations |
| B. | Q1 Kit Configuration |

| | |
|---|---|
| 1 | Inventory of Kit Content |
| 2 | How to Use |

| | |
|---|---|
| a. | Unpack and Identify Content |
| b. | Assess Sampling Locations |
| c. | Filling Out Sampling Report |
| d. | Sampling Instructions (Step by Step) |
| e. | Repacking Kit for Shipment |

| | |
|---|---|
| 3 | Return Shipping Instructions |
| 4 | Test Results |

| | |
|---|---|
| C. | Q2 Kit Configuration |

| | |
|---|---|
| 1 | Inventory of Kit Content |
| 2 | How to Use |

| | |
|---|---|
| a. | Unpack and Identify Content |
| b. | Assess Sampling Locations |
| c. | Filling Out Sampling Report |
| d. | Sampling Instructions (Step by Step) |
| e. | Repacking Kit for Shipment |

| | |
|---|---|
| 3 | Return Shipping Instructions |
| 4 | Test Results |

| | |
|---|---|
| D. | Q3 Kit Configuration Options |

| | |
|---|---|
| V. | Toxic Hazards |

| | |
|---|---|
| A. | Responsible Risk Management |
| B. | Remediation Options |
| C. | Calculating Cleanup Costs |
| D. | Selecting a Contractor |
| E. | Do-It-Yourself Resources |

| | |
|---|---|
| VI. | Relief Resources |

| | |
|---|---|
| A. | Crime Victim Assistance Resources |
| B. | Property Insurance Coverage |

| | |
|---|---|
| VII. | Miscellaneous Information |

The training formats for this audio visual component will include standard instructional media presentations in both DVD and VHS formats as well as on-line, web-based video media formats such as presentations prepared using Quicktime, Windows Media, and other such electronic audio-visual on-line video presentation formats. Additionally, printed instructional manuals will also be included within the Kit a basic procedural reference and reinforcement aid as well as a supplemental information resource.

Narcotics Surface-Deposit Residue Sampling Process

The Clan-Lab Home Test Kit includes a system, protocol, method and materials for instructing an individual of average intelligence in the practice of taking wipe samples from suspect surface areas and returning said samples to the Kit provider or to another specified specialty laboratory location for analysis measurement of trace narcotic residues.

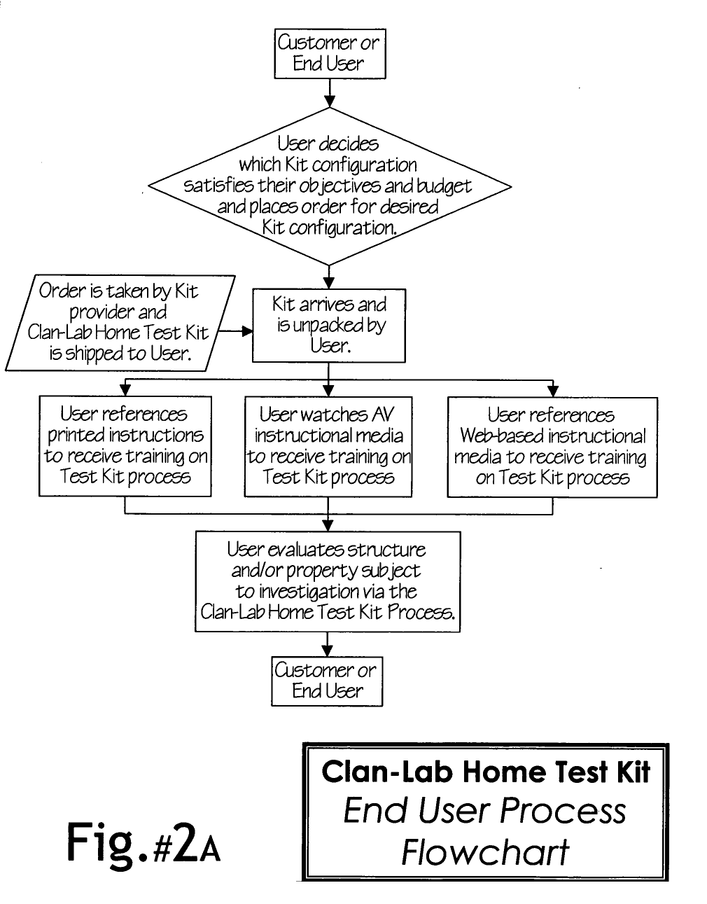

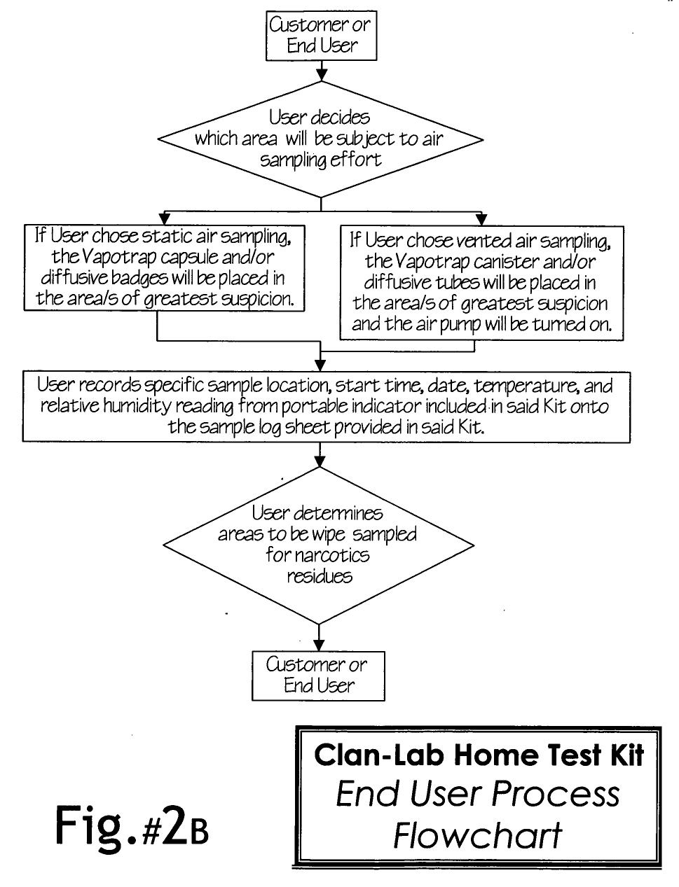

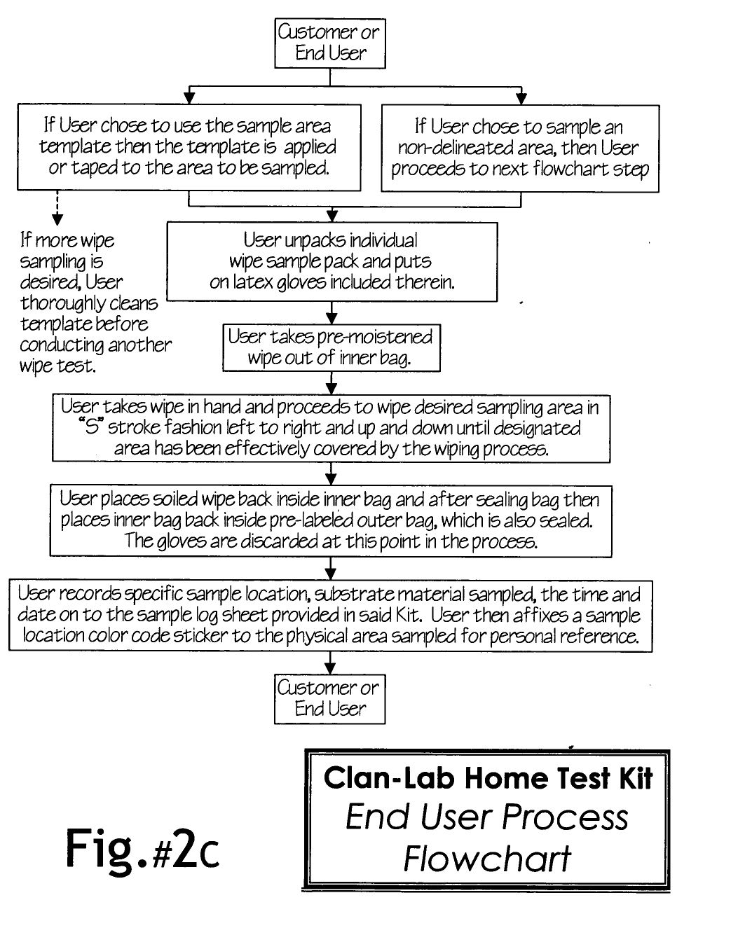

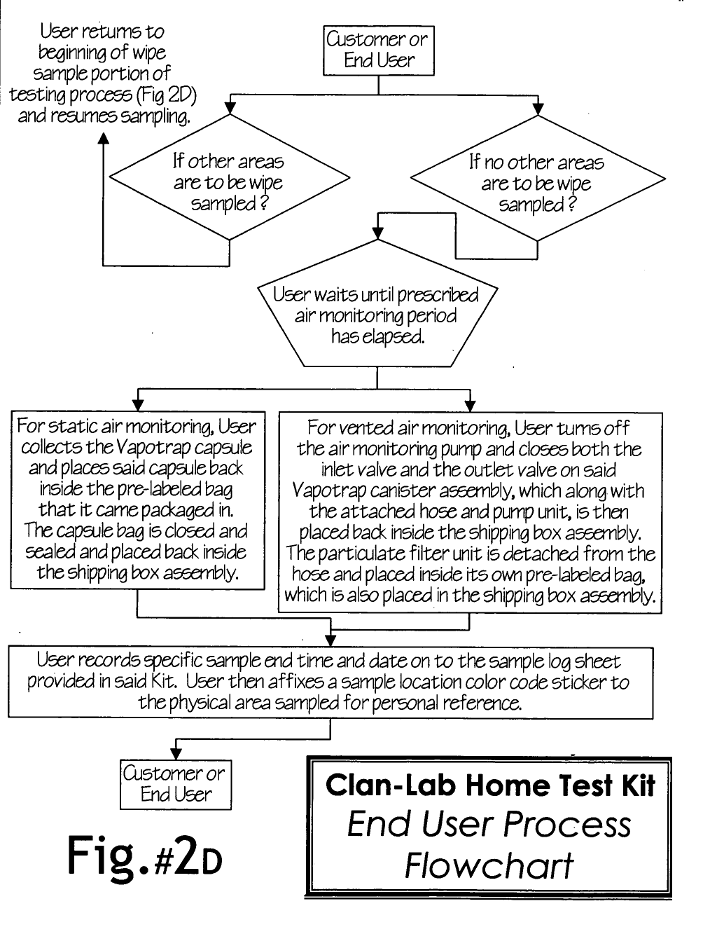

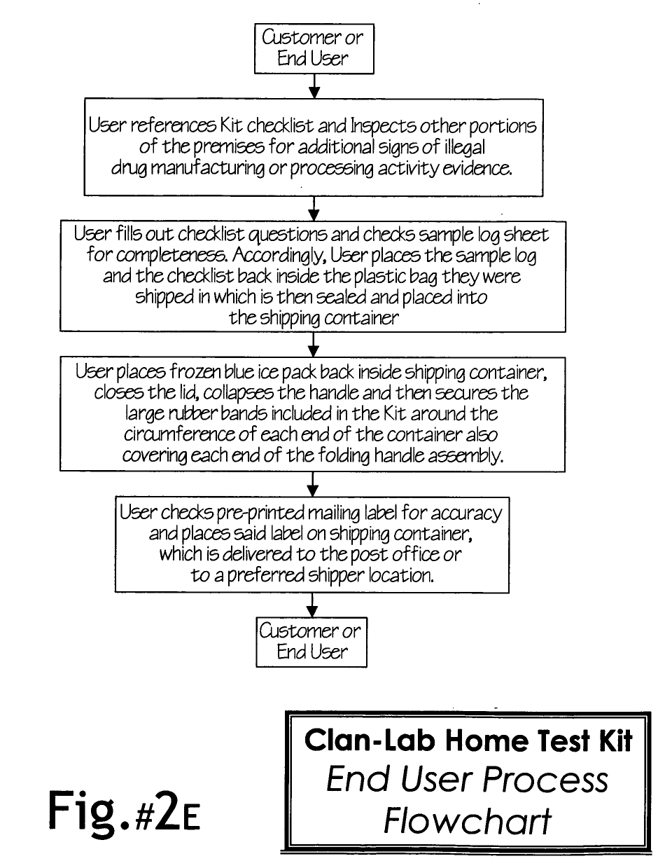

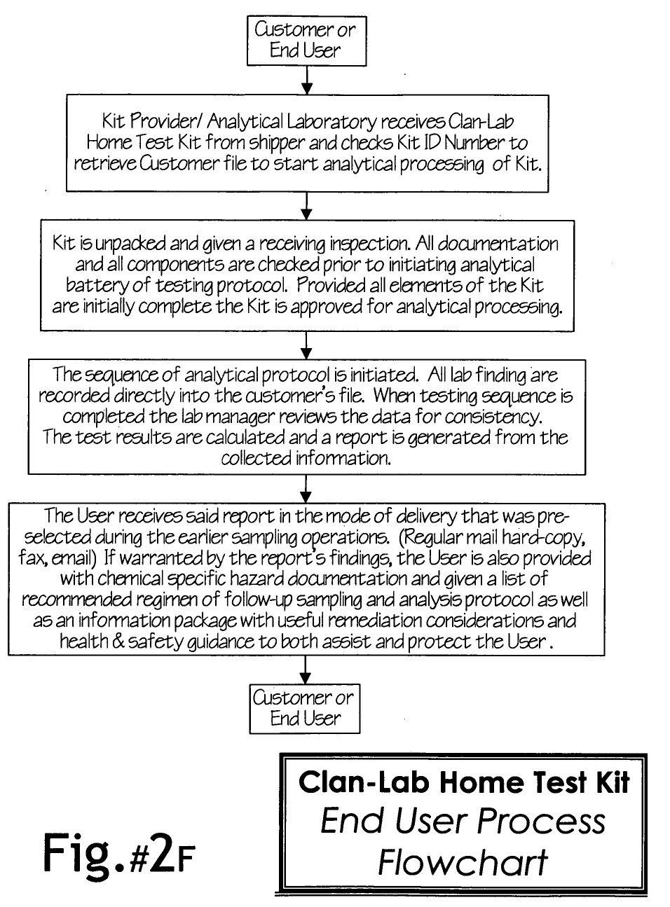

The step-by-step procedure for this aspect of the embodiment is as follows:
1) The Kit user reviews in advance the DVD disk or VHS tape included in said Kit and reviews the written procedure as a point of reference and review prior to initiating physical sampling activity. FIG. 2A demonstrates the process by which the user is trained through the instructional media component of said Kit and thereby gains a competency to perform said sampling procedure.
2) Using the knowledge gained through the instructional media training component of the Kit and after physically inspecting said premises for the most likely locations said clandestine drug manufacturing acts may have occurred, the Kit user selects the wipe sample test pack from the Kit's shipping container and opens said sampling pack. Portions of FIG. 2B, all of FIG. 2C, and portions of FIG. 2D illustrate the operational flow of the wipe sampling process and how this series of individual test relate to the comprehensive function of the Kit as a whole.
3) After putting on a pair of disposable gloves included in the Clan-Lab Home Test Kit, the individual user would take a pre-packaged wipe* from its sealed package and wipe a desired section of the suspect area or device and then place the wipe inside a pre-marked, color-coded, plastic bag for the wipe and seals the bag accordingly. The user will follow the detailed instructions given in the Kit's instructional media pack, which details the proper techniques for using the Kit's wipe test packages to properly take representative samples across sections of a structures wall, floor, ceiling, or other interior surface areas (* Depending upon the chemistry of the suspected or known clandestine laboratory operation or that of similar labs discovered in the vicinity of the investigated premises, the Kit provider may elect to provide either a dry wipe or to pre-saturate said wipe with a fluid substance that may include water, a surfactant solution, alcohol, or other solvent material.)
4) The individual would then discard the gloves after taking each sample to avoid possibility of cross contaminating other samples.
5) The used wipe sample bag is then placed inside the larger sample bag, which originally contained both the wipe sample bag and the gloves, and sealed accordingly.
6) A notation is made on the sample log sheet identifying the respective wipe sample identification code as well as the location, type of substrate sampled, and the estimated area dimensions covered by the sample.
7) The sealed sample pack bag is placed in the return-mailing container along with the other tests required in the Clan-Lab Home Test Kit.
8) When all tests required in the Clan-Lab Home Test Kit are completed the User inspects said Kit for all items of sampling equipment and/or samples and double checks the Kit's documentation for completeness. The Kit is then closed, sealed, labeled, and shipped back to the Kit provider or the designated laboratory for analytical processing of the samples contained therein.
9) The returned sample will be analyzed at a specially prepared laboratory facility and the results from said analysis will be communicated to the individual via the method of contact requested by the user. This test will yield qualitative and/or quantitative evidence of the presence or absence of narcotics residue when analyzed.

In the Narcotics Qualification Process, this invention promotes using a selection of electronic detection methodologies for the analysis of this Kit component. Along with the expected degree of variability in clandestine drug manufacturing chemistry, there is also significant degree of variability in vapor pressure and vapor concentration in illicit narcotic substances. For instance in four major and relatively commonplace drug substances such as methamphetamine, cocaine, heroin, and LSD, the vapor concentrations vary by more than eight orders of magnitude. Whereas methamphetamine at normal temperatures has a vapor concentration of over 200 parts per million, heroin only has a vapor concentration of 1 part per trillion. Likewise, LSD has a vapor concentration only slightly higher than heroin and cocaine is only a fraction of one part per billion.

The nature of clandestine drug manufacturing introduces a wide degree of homemade drug recipes, which contributes to variability even within the same type of narcotic substance. As a general consideration, it is recognized that a temperature increase of 9° F. or 5° C. will approximately double the amount of vapor that is present at equilibrium above a solid compound at or near room temperature. In effect, this means that when the ambient temperature rises by heating an object that is suspected of containing illicit drugs an increasing amount of vapor will be present for detection. This invention varies from other narcotics wipe sample approaches by using several type of drug detection apparatus and benefiting from the accuracy one detection methodology has over another in the investigation of a given narcotic substance.

One skilled in the art of electronic drug detection practice will appreciate this invention's flexibility and novelty by not restricting its analytical resource selection to that of one technology provider. This novel approach to investigating narcotic wipe samples results represents a new and useful improvement to any singular electronic detection methodology by yielding fewer false positive results and thus adds an element of quality assurance confirmation; in that, some samples will be processed by more than one detection methodology (given the particular type of suspected clandestine laboratory chemistry thus indicated by other evidence and samples taken from the premises subject to the investigation effort).

The narcotics detection technology chosen by this invention is based upon the selective ion mobility principle; whereas, one or more of the following drug detection technologies will be employed to ascertain the identity of the potential narcotics residues collected upon said wipe sample tests as per this aspect of the Narcotics Qualification Process. The analytical test protocol will employ one or more of the following detector types:
a.) Ion Mobility Spectrophotometer (IMS)*
b.) Surface Ionization Detector (SID)*
c.) Differential Mobility Spectrophotometer (DMS)*
d.) Field Ion Spectrophotometer (FIS)*
e.) Surface Acoustic Wave Detector (SAW)*
f.) Raman Spectrophotometer (RS)
* May be subject to analytical configuration as a recipient of a pre-separated exit gas flow from a gas chromatograph.

In instances where the end user requests a quantitative analysis of the narcotics detection wipe samples a Gas Chromatograph Mass Spectrophotometer (GCMS) may be employed either acting solely or in conjunction with one of the previously identified detection technologies; whereas, an optional Doplant or Carrier Gas may be employed to a sure that the detector is functioning appropriately and that the suspected narcotics substance peak signature is contextualized to a standard. In this scenario, the Kit user will be provided with a template to be taped or affixed to said area being sampled to restrict the sample to a given dimension, which will expressed in a units of contaminate detected per given square area format.

Airborne Residual Chemical Concentration Testing

The Clan-Lab Home Test Kit includes a system, protocol, method and materials for instructing an individual of average intelligence in the practice of taking samples from suspect indoor air atmosphere and returning said samples to the Kit provider or to another specified specialty laboratory location for analysis measurement of airborne residual chemical concentration or contamination residues.

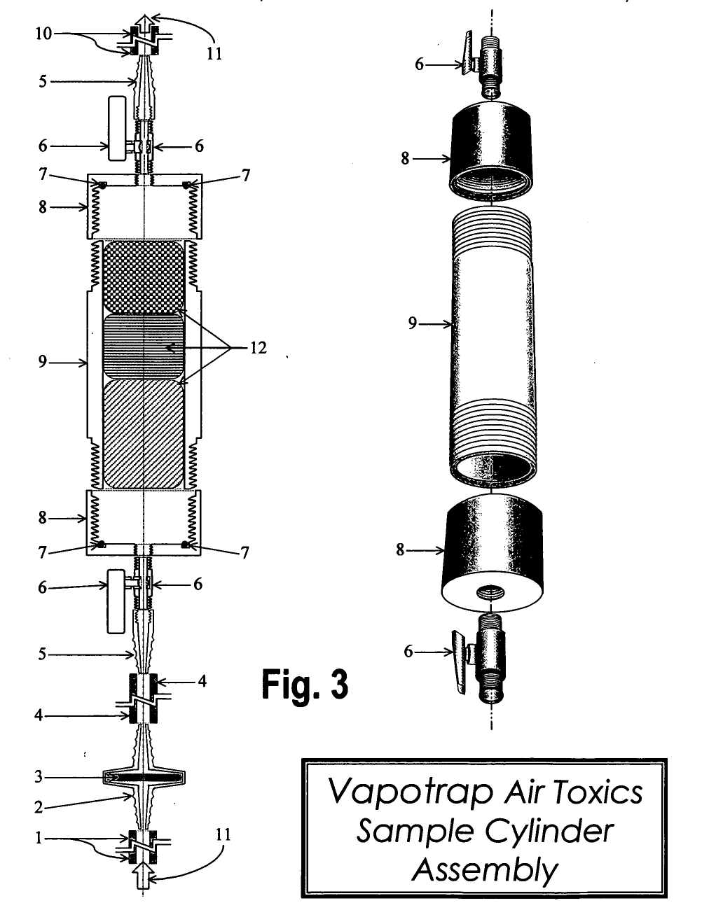

The step-by-step procedure for this aspect of the embodiment is as follows:
1) The Clan-Lab Home Test Kit user reviews in advance the DVD disk or VHS tape included in said Kit (FIG. 1) and reviews the written procedure as a point of reference and review prior to initiating physical sampling activity. FIG. 2A demonstrates the process by which the user is trained through the instructional media component of said Kit and thereby gains a competency to perform said sampling procedure.
2) Using the knowledge gained through the instructional media training component of the Kit and after physically inspecting said premises for the most likely locations said clandestine drug manufacturing acts may have occurred, the Kit user selects the air sample test equipment elements from the Kit's shipping container and opens said sampling pack. FIG. 2B and portions of FIG. 2D illustrate the operational flow of the air sampling process and how this individual test activity relates to the comprehensive function of the Kit as a whole.
3) After putting on a pair of disposable gloves included in the Clan-Lab Home Test Kit, the individual user would take the air sampling device and place said device in the area most likely to have been the site of suspect drug making activity or other such area that has yielded evidence of such contamination possibilities. The air-sampling device should be placed in a “worst case” location low to the floor in said suspect area; whereas, the vapor density of many chemicals used in the clandestine drug making process are heavier than air and, as such, low areas are where children often play and would be subject to the greatest exposure risks from this manner of contamination hazard.
4) If the particular air-sampling device selected by the Kit user is a static device, which relies solely upon the diffusive principle of adsorption or absorption, the device is placed in the area most likely to be impacted by a potential chemical influence. In like manner, if the particular air-sampling device selected by the Kit user is a vented or powered air-sampling device, which relies a flow of air being forced or drawn through a diffusive adsorption or absorption media or media collection, the device is placed in the area most likely to be impacted by a potential chemical influence and the integral or attached air pumping or vacuum system is turned on to begin the sampling event.
5) The Kit user, with the instruments, materials, and documentation provided in said Kit (FIG. 1), then records the time, date, location, mode of sampling selected, temperature and relative humidity of the conditions at the time the sampling event occurs on the sample log sheet along with an estimate of the area dimensions covered by the sample.
6) When the allotted time period for the sampling event is completed (FIG. 2D), as specified by the Kit provider, the user retrieves the air sampling device and/or sampling equipment and returns said equipment to the Kit's shipping container.
7) If a static air-sampling device was employed, said device is then returned to its pre-marked, color-coded sample container and also placed inside said Kit's shipping container.
8) If a vented air-sampling device was employed, said device is:
   a) unloaded of its sampling media, which is then returned to its pre-marked, color-coded sample container and also placed inside said Kit's shipping container along with the pump and ancillary connecting hose and associated equipment apparatus; or
   b) the inlet and outlet valves of the Vapotrap Canister assembly (FIG. 3) are shut and the device along with the medias contained therein are placed inside said Kit's shipping container along with the pump and ancillary connecting hose and associated equipment apparatus.
9) The Kit user, with the instruments, materials, and documentation provided in said Kit, then records the time, date, temperature and relative humidity of the conditions at the time the sampling event ends on the sample log sheet.
10) When all tests required in the Clan-Lab Home Test Kit are completed the User inspects said Kit for all items of sampling equipment and/or samples and double checks the Kit's documentation for completeness. The Kit is then closed, sealed, labeled, and shipped back to the Kit provider or the designated laboratory for analytical processing of the samples contained therein.
11) The returned sample will be analyzed at a specially prepared laboratory facility and the results from said analysis will be communicated to the individual via the method of contact requested by the user. (FIGS.: 4, 5, & 6) This test will yield qualitative and/or quantitative evidence of the presence or absence of airborne chemical contamination when analyzed.
   Vapotrap Capsule

The Vapotrap Capsule represents a novel device, system, and method for qualifying and/or quantifying chemical substance concentrations from an atmosphere.

The device can absorb and/or absorb airborne contaminants and gasses through the diffusion process from either a normal, static convectional airflow or the device can be used with a forced flow of air being introduced into said capsule. Obviously, the static mode of sampling is simpler to perform and takes a long sampling period than the more efficient vented method. FIG. 2B shows an operational flowchart sequence the Clan-Lab Home Test Kit process and how this embodiment fits into the inspection process.

The Capsule device itself consists of a breathable mesh bag or pouch with a sealing mechanism to prevent its contents from becoming displaced. In a typical configuration, the Vapotrap Capsule contains three to five smaller capsules or sub-pods, each containing a pre-measured unit of adsorbent or absorbent media. In like manner, the subpods are constructed of a natural or synthetic porous mesh material to allow for unrestricted ventilation within the adsorbent and/or absorbent medias contained therein; whereas, each mesh packet or pod has a sealing component to:
(a) prevent the spillage of the individual adsorbent and/or absorbent media material contained therein,
(b) allow for respective sub-pod to be opened and emptied as may be deemed necessary given the circumstances and facts available relative to the premises being investigated; whereas, an alternative analytical process may become necessary due to the particular challenges presented by the clandestine chemistry considerations by the test location, and
(c) allow the subpods to be either emptied or filled by the receiving or supplying laboratory personnel and/or recycled through desorption or the disposal of the same.

The specific content of the Vapotrap Capsule is dictated by the physical and chemical parameters of the application; whereas, the use of various types of media can more effectively capture and retain a wider range of chemical substances than any single media can accomplish. This adsorption performance flexibility is very effective for monitoring airborne contaminants related to past or present clandestine laboratory activity, given the enormous range of diverse chemical ingredients, which are known to be used in this unique and dangerous criminal enterprise.

The Vapotrap Capsule is generally comprised of at least two or more sub-capsules (or subpods), each being individually filled with a certain type, grade and mesh size of separate elements of adsorbent and/or absorbent medias specially selected by the Kit provider based upon information that may have been provided by the end user, law enforcement, and/or regionally observed trends in clandestine drug chemistry. The composite Capsule unit serves the purpose of collecting airborne contaminants from either a static or forced airflow within a structure's interior or indoor atmosphere and includes at least two or more of the following components:
a) activated carbons,
b) zeolites,
c) organic polymers,
d) metal chlorides,
e) silicates,
f) sulfates,
g) silicas, and/or
h) aluminas.

The isotherm capacity for any particular media form is the numerical coefficient of its relative adsorption strength. There are a number of factors that influence a media's capacity to adsorb or absorb chemical compounds. The media itself for instance, even within various grades of the same media substance there is a substantial variability per given chemical. Activated carbon for instance, is available in a variety of grades with different properties, pore sizes, and affinities for adsorption of contaminants. Other factors also come into play in the adsorption process such as the type and concentrations of chemicals present in the atmosphere, the temperature and relative humidity, as well as the time allotted for the testing episode or residence time.

From an activated carbon perspective, it is generally recognized that chemical compounds are good candidates for adsorption provided that they have a molecular weight above 50 and a boiling point greater than 50° C. The nature of clandestine drug manufacturing is such that many different types of compounds are blended, cooked and synthesized by “cookers” with little or no chemistry background and most often with a reckless disregard for consequences that themselves or others may have to face for their acts. Accordingly, the supplies of ingredients range from whatever they can buy off the shelf or steal from commercial or industrial sources; therefore, no hard and fast rules apply to the clandestine drug manufacturing process and activated carbons alone do not possess the capacity and flexibility necessary to keep pace with this problem.

In today's information age, an illegal drug recipe or manufacturing technique can theoretically be published on an internet website one week and be put into practice on worldwide basis within a matter of days. The novelty of the approach presented in this embodiment of using multiple adsorption medias in a sub-pod arrangement is such the composite adsorption capsule can be reformulated rapidly enough to meet the elusive challenges presented by this unique hazard and problem to society.

Another advantage to the embodiment of this invention is its ability to be formulated with a media selection to account for the range of temperature anticipated for the sampling event. At a given airborne chemical concentration, temperature changes from 32° F. to 140° F. can impact the adsorption capacity of some medias several orders of magnitude. The vapor pressure of a chemical substance is always a function of temperature and increases exponentially with increasing temperature. Again because of the unusual characteristics of the application pursuant to the intent of this invention, sampling temperature cannot always be adjusted to the ideal; whereas, this invention can be custom prepared for whatever range of temperatures are anticipated to be premises subject to investigation.

Still another advantage to the embodiment of this invention is its ability to benefit from the dynamics of one media's ability to retain and preserve a certain chemical compound over that of another. For instance, many times activated carbon will adsorb a given reactive solvent substance, such as acetone, methyl ethyl ketone, or styrene, and in doing so will begin to catalyze its decomposition. By incorporating other adsorption medias into the Vapotrap capsule, such as organic polymer medias for example, this type of catalyzation does not occur at significantly measurable levels and the true airborne contaminant ratios subject to the investigation effort will be reported without being as dramatically distorted as an adsorption based assessment solely dependant upon using activated carbon media alone. Therefore, it is another embodiment of this invention that multiple grades and types of adsorbent and absorbent compounds are used in unison to more effectively capture, preserve, and retain chemical substances and effectively release said substances during a subsequent extraction, measurement, and analysis process.

It is a well known fact, that the clandestine laboratory issue is an unsolved national problem that has hazardous impact potential that is as yet undefined. Several legislative initiatives are underway at present trying to stimulate the development of science and gain an understanding of these problems. One such example can be referenced in a recently introduced bill (H.R. 798 & S.2019) otherwise known as the “Methamphetamine Remediation Research Act of 2005” which seeks to “provide for a research program for remediation of closed methamphetamine production laboratories, and for other purposes.”

One such embodiment of this invention is that significant portions of the aforementioned problem can be discovered and solved and while the problem being studied in unison. It is an inherent characteristic of this invention that it has an ability to benefit from the perpetual research opportunities offered as a synergistic bonus to the immediate advantage the Clandestine Laboratory (or Clan-Lab) Home Test Kit presents as an economical solution that is made available to the general public at large. As a means of determining hidden hazards presented by past or present clandestine manufacturing or processing activities, the Clan-Lab Home Test Kit can perform its primary function and develop, by means of the component embodiment presented in the Vapotrap Capsule, a significant amount of strategic scientific information about the national impact this manner of crime is causing. Whereas, the Vapotrap Capsule offers opportunities to study a problematic adsorption application that has, through its wide variety of clandestine chemistries, defied the conformity necessary for predictive adsorption modeling and has thus allow the problem to evade scientific understanding for over a decade while hundreds of thousands of innocent persons have been impacted to some degree.

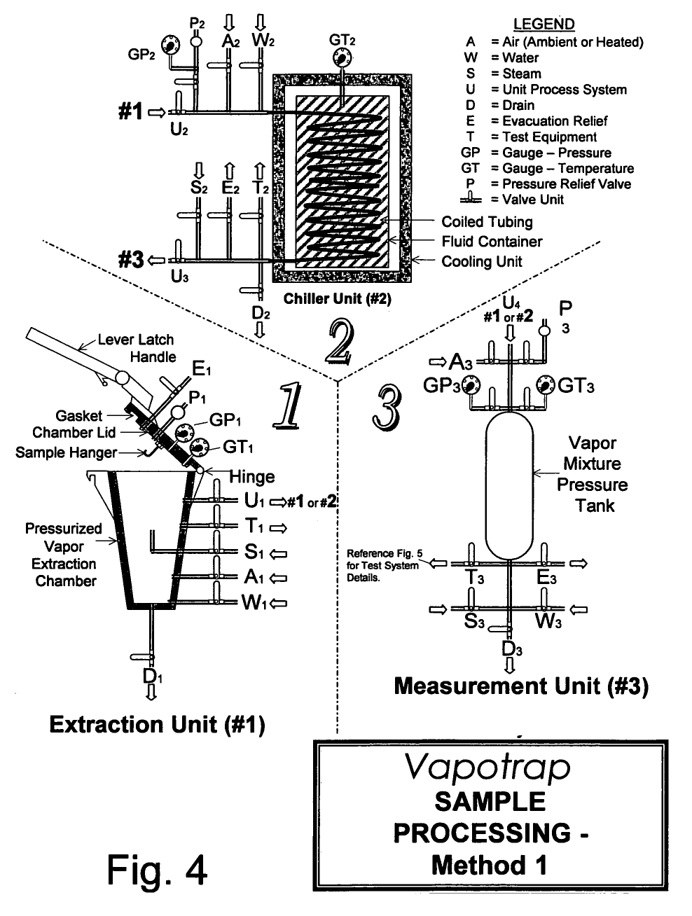

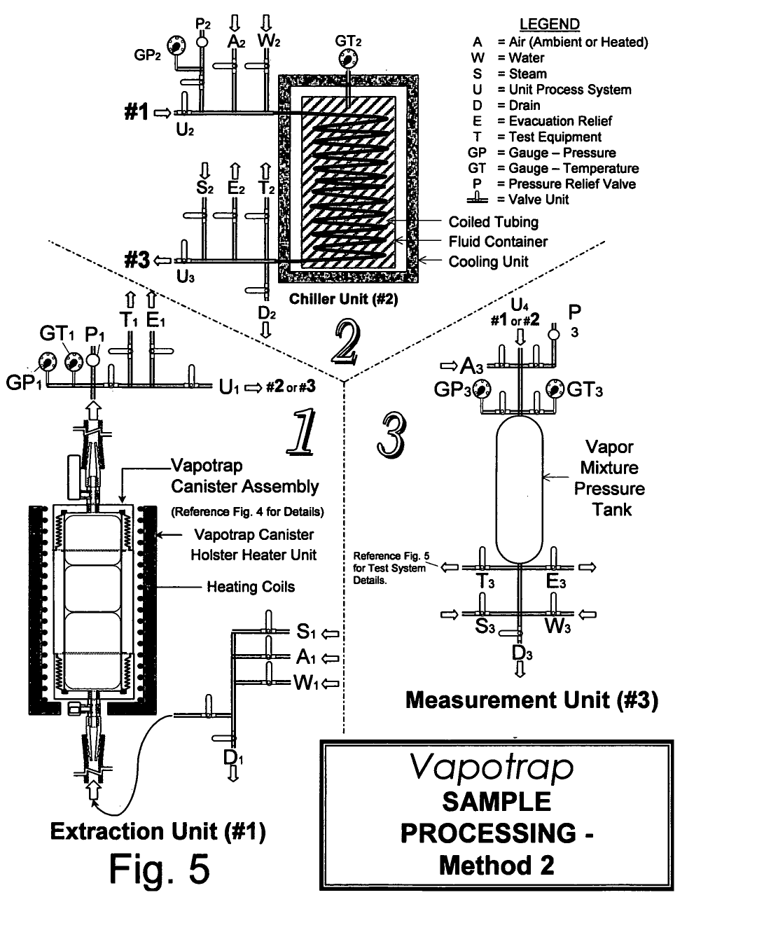

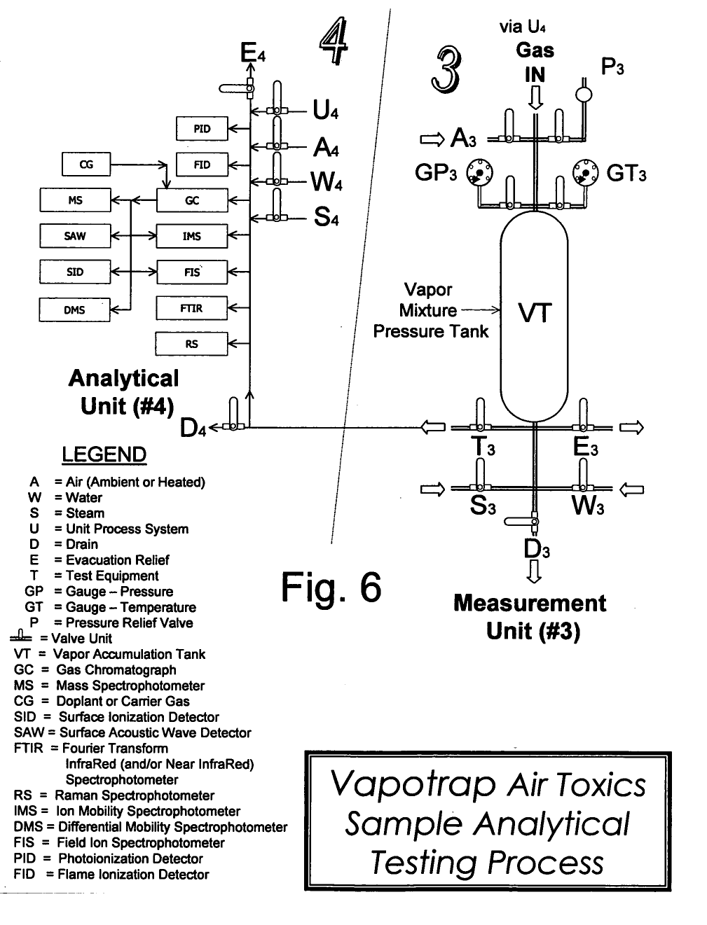

The Vapotrap Capsule, when used as a static monitoring device or when specific conditions relative to the particular investigation so warrant, the returned Capsule assembly is prepared for analysis as follows:
a) removing said sample from shipping container and cross referencing the attached identification label thereto to the sample log sheet also retrieved from said sample shipping container;
b) entering the appropriate sample identification code into the customer or end users account file;
c) removal of said sample from its container packaging;
d) placing said sample inside an air tight Extraction Chamber apparatus (FIG. 4—Extraction Unit Detail, Drawing Segment 1) and sealing said chamber;
e) introducing a heat and/or steam source to said Extraction Chamber (FIG. 4—Extraction Unit Detail, Drawing Segment 1, Valve S₁);
f) opening the outlet valve located on said Extraction Unit assembly once the desired temperature, pressure, and time period are achieved (FIG. 4—Extraction Unit Detail, Drawing Segment 1, Valve U₁);
g) routing off-gas flow directly from said Extraction Chamber into the Vapor Expansion/Mixing Tank assembly (FIG. 4—Measurement Unit Detail, Drawing Segment 3, Valve U₄); or routing said off-gas flow indirectly into a Vapor Expansion/Mixing Tank via an attached Chilling Unit assembly (FIG. 4—Chilling Unit Detail, Drawing Segment 2, Valve U₂) designed to reduce the temperature of the off-gas flow as it passes through a length of chilled tube or pipe;
h) opening a valve on the Vapor Expansion/Mixing Tank network (FIG. 4—Measurement Unit Detail, Drawing Segment 3, Valve T₃) leading to the analytical instrumentation network (FIG. 6—Analytical Unit Detail, Drawing Segment 4);
i) recording the measurements observed into the end user's account file;
j) releasing all residual vapors that may be contained said Extraction Chamber, Chilling Unit, and/or Vapor Expansion/Mixing Tank or the associated piping thereto;
k) removing sampling media (Capsule or Subpod) from the Extraction Chamber and re-sealing the lid assembly; and
l) purging the remainder of the Extraction, Chiller, Measurement, and Analytical systems with high temperature steam and/or water flow for cleaning prior to processing another sample Capsule and/or Subpod assembly for analysis.
   Vapotrap Canister

Although the Vapotrap Capsule can be used as a static monitoring device, which simply adsorbs chemical substances by diffusion through normal indoor convectional air currents, the dynamics of moving a flow of ambient air through the medias contained therein is more efficient process when forced ventilation is applied. The Vapotrap Canister offers a variety of embodiment advantages as a component to the Clan-Lab Home Test Kit (FIG. 1). FIG. 3 illustrates one form of the embodiment of this invention.

The Canister device itself (FIG. 3) consists of four primary mechanisms (Reference Table 3 for inventory of apparatus componentry):

First, a chamber (FIG. 3—Article 9) that serves to: a) hold the Capsule (FIG. 3—Article 12) of segregated medias thus allowing a flow of gas (FIG. 3—Article 11) to be passed through said medias, b) act as a housing for the media and function not only in a sampling capacity, but also in an analytical capacity (FIG. 5); whereas when a metallic version of the chamber is employed in the investigation process, the media does not have to be unloaded at the analysis laboratory, but rather can function as an extraction apparatus and contain both the pressures and temperatures necessary for thermal desorption of the contaminants contained within the spent media Capsule.

Second, one or more lids or sealing mechanisms (FIG. 3—Articles: 7 and 8) that:
a) can be opened for extracting and replacing said Capsule (FIG. 3—Article 12),
b) can be sealed to prevent escape, bypass or short-circuiting of gas flow (FIG. 3—Article 11) around the Capsule's media components (FIG. 3—Article 12),
c) can be opened and/or disassembled for cleaning and sterialization purposes, and
d) can contain the internal steam pressures generated by the thermal desorption process.

Third, an inlet and outlet valve assembly (FIG. 3—Article 6) that:
a) can be to control air flow (FIG. 3—Article 11) to and from the Canister assembly,
b) can allow the Canister to be closed securely prior to and immediately after sampling to lock in and prevent the escape of collected contaminants and eliminate the need, unnecessary bulk, and inconveience for another container, and
c) can allow for the desorbed contaminants to be controlled and released as required in the subsequent analytical process.

Fourth, a pump, hose, and hose attachment fitting assembly (FIG. 3—Articles: 1, 4, 5, and 10) that:
a) can allow the Canister to either have an air flow (FIG. 3—Article 11) forced through it by pressure or drawn through it by vacuum,
b) can allow the use of an inline particulate filter assembly (FIG. 3—Articles 2 and 3) to capture airborne particles for subsequent analysis,
c) be readily disconnected as the Canister is reattached to the analysis network and used as an extraction chamber (FIG. 5—Extraction Unit Detail), and
d) can be inspected, cleaned, and reattached to a freshly prepared Canister, shipped to an end user and reused again as a ventilation component in another sampling event.

Another embodiment of this invention is the flexibility of being able to configure a lower cost, lower weight capsule assembly apparatus, which assembled from synthetic plastic and/or fiber substrate materials and performs essentially the same in a sampling mode. Conversely, analytical processing of this variation is accomplished by opening the Canister at the receiving laboratory facility; whereas, the said Capsule assembly content is then emptied from said Canister and transferred into a heat resistant Canister assembly (made of metallic components) which is then placed in a heating source holster assembly (FIG. 5—Extraction Unit Detail) to facilitate direct analysis of the contaminants contained in the adsorbent and/or absorbent components contained therein and thus extract an off-gas flow via the thermal desorption process.

The Vapotrap Canister assembly is prepared for laboratory analysis by:
(a) removing said sample canister assembly from shipping container and cross referencing the attached identification label thereto to the sample log sheet also retrieved from said sample shipping container;
(b) entering the appropriate sample identification code into the customer or end users account file;
(c) placing said sample canister assembly inside a heated yoke assembly for vapor extraction processing (FIG. 5—Extraction Unit Detail, Drawing Segment 1);
(d) connecting system hoses to said canister assembly and opening both the inlet valve;
(e) introducing a heat source to the heating holster assembly unit containing or enveloping the prepared Canister assembly (FIG. 3);
(f) introducing a pressure, heat, and/or steam source to said canister assembly (FIG. 5—Extraction Unit Detail, Drawing Segment 1, Valve S₁);
(g) opening the outlet valve located on said Extraction Unit assembly once the desired temperature, pressure, and time period are achieved (FIG. 5—Extraction Unit Detail, Drawing Segment 1, Valve U₁);
(h) routing off-gas flow directly from said Extraction Chamber into the Vapor Expansion/Mixing Tank assembly (FIG. 5—Measurement Unit Detail, Drawing Segment 3, Valve U₄); or routing said off-gas flow indirectly into a Vapor Expansion/Mixing Tank via an attached Chilling Unit assembly (FIG. 5—Chilling Unit Detail, Drawing Segment 2, Valve U₂) designed to reduce the temperature of the off-gas flow as it passes through a length of chilled tube or pipe;
(i) opening a valve on the Vapor Expansion/Mixing Tank network (FIG. 5—Measurement Unit Detail, Drawing Segment 3, Valve T₃) leading to the analytical instrumentation network (FIG. 6—Analytical Unit Detail, Drawing Segment 4);
(j) recording the measurements observed into the end user's account file;
(k) releasing all residual vapors that may be contained said canister assembly, chilling unit, and/or vapor expansion/mixing tank or the associated piping thereto;
(l) removing canister assembly from the heating yoke unit (FIG. 5—Extraction Unit Detail, Drawing Segment 1), empty sampling media Capsule from therein and subject said Canister assembly (FIG. 3) to disassembly, cleaning, drying, and reloading with sanitized, prepared composite media Capsule before being redeployed; and

(m) purging the remainder of the Extraction, Chiller, Measurement, and Analytical systems with high temperature steam and/or water flow for cleaning prior to processing another sample Canister assembly for analysis.

TABLE 3
Clandestine Laboratory (Clan-Lab) Home Test Kit
Vapotrap Canister Assembly

| No. | Item | Units | Description | Function |
|---|---|---|---|---|
| 1 | Hose | 1 | Synthetic plastic (variable sized | Inlet air flow |
| | | | diameter) | |
| 2 | In-line Filter | 1 | Synthetic plastic (variable sized orifice) | Container for filter element (3) |
| | Assembly | | | |
| 3 | Filter Element | 1 | Synthetic (variable sized micron mesh | Filtration of particulates |
| | | | and hose diameters) | |
| 4 | Hose | 1 | Synthetic plastic (variable sized | Routing air flow into Vapotrap cylinder |
| | | | diameter) | assembly |
| 5 | Hose Nipple | 2 | Metallic or synthetic plastic (variable | Attaching hose to Vapotrap cylinder |
| | | | sized threading and hose diameter) | assembly |
| 6 | Shutoff - Ball | 2 | Metallic or synthetic plastic (variable | Controlling air flow |
| | Valve | | sized threading and orifice diameter) | |
| 7 | O-Ring Gasket | 2 | Synthetic plastic (variable diameter and | Sealing Vapotrap cylinder chamber (9) |
| | | | thickness) | cap (8) seating |
| 8 | Cap Assembly | 2 | Metallic or synthetic plastic (variable | Cap for Vapotrap cylinder (9) and |
| | | | sized threading and diameter) | bushing for attachment of Shutoff - Ball |
| | | | | Valve (6). |
| 9 | Cylinder Body | 1 | Metallic or synthetic plastic (variable | Container for Vapotrap capsule and |
| | | | sized threading and diameter) | subpods (12) |
| 10 | Hose | 1 | Synthetic plastic (variable sized | Outlet air flow |
| | | | diameter) | |
| 11 | Air flow | NA | Variable flow rate given air | Transport medium for airborne |
| | | | pump/vacuum volume and velocity | contaminates into Vapotrap cylinder |
| | | | regulated by diameter and resistance. | assembly and allow for filtered exit gas |
| | | | | to depart from the same. |
| 12 | Vapotrap | Varies | Various adsorption media packets | Adsorb and contain airborne |
| | capsule and | | | contaminates from air flow (11) |
| | subpods | | | |

1. The method of providing a pre-assembled, comprehensive sampling Kit, herein referred to as the Clandestine Laboratory (Clan-Lab) Home Test Kit, to allow for the sampling and qualitative and/or quantitative analysis of gaseous, liquid, and/or solid samples related to the discovery of toxic chemical hazards, which may be associated with past or present illegal clandestine drug manufacturing or processing activities;
wherein said activities may have contaminated the subject structure and/or the real estate premises.

2. A means of preparing and providing a Clandestine Laboratory (Clan-Lab) Home Test Kit to be used in testing for a plurality of residual airborne and surface deposit contaminants as well as other forms of contamination related to past or present illegal clandestine drug manufacturing or processing activities; whereas private citizens, tenants, property owners, crime victims, law enforcement officers, child protection workers, public health officials, media personnel, representatives of the court systems, home inspectors, health and safety officials, environmental officials, municipal workers, park rangers, academic and research personnel, and other government or military personnel can use said Kit to safely test suspect premises or properties to identify, distinguish, and measure evidence of chemical hazards pursuant to suspected, or known, former illegal drug manufacturing and/or processing activities.

3. The use of a porous fabric capsule, which is thereby comprised of individually segregated porous fabric sub-capsules (or subpods), each being individually filled with a certain type, grade and mesh size of separate elements of adsorbent and/or absorbent medias for the purpose of collecting airborne contaminants from either a static or forced airflow within a structure's interior or indoor atmosphere pursuant to the subsequent analysis of said contaminants via the off-gasses released from thermal desorption processes; whereas said capsule includes at least two or more of the following components:
a) activated carbons,
b) zeolites,
c) organic polymers,
d) metal chlorides,
e) silicates,
f) sulfates,
g) silicas, and/or
h) aluminas.

4. A system and protocol for a Clandestine Laboratory (Clan-Lab) Home Test Kit to be used for testing for a plurality of residual contaminants, including airborne, surface deposit, surface water, groundwater, soil, and suspect unknown solid or liquid substances as well as testing for said residual contaminants that may have absorbed into structural materials, furnishings, and objects of personal property such as clothing or electronics; whereas said residual contaminants are related to past or present illegal clandestine drug manufacturing or processing activities.

5. A method according to claim 4 whereas the audio-visual instruction media, as well as the printed documentation components, are custom designed to enable an individual of average intelligence to proficiently conduct the designated tests and the associated collection of samples heretofore generated during the said on-site testing process.

6. The method of claim 4 wherein said step of taking samples comprises sampling for narcotics residues as well as organic and inorganic compounds in suspect solid, liquid, or gaseous medias pursuant to a past or present clandestine laboratory (Clan-Lab) operation involved in the illegal manufacturing or processing of narcotics substances including the associated precursor and recursor substances incidental to said narcotics manufacturing or processing activities in addition to the ingredients thereof and/or the waste products thereto, all of which collectively are herein referred to as “drug” or “drugs”.

7. A process according to claim 4; wherein the procedure of detecting, qualifying, quantifying, and providing notification of an environmental hazard, pursuant to residual contamination originating from a past or present clandestine drug manufacturing or processing operation, thereby comprises a field testing Kit, to be employed by the end user or customer, and returned by the same to a designated analytical laboratory specially equipped, trained, and configured for the unique assessment application pursuant to the purpose of this invention; whereas a report is generated by said analytical laboratory and communicated to the Kit user or customer.

8. A system and protocol according to claim 1; wherein a Clan-Lab Home Test Kit is provided for an environmental impact inspection and assessment (pursuant to residual contamination caused by clandestine drug manufacturing and/or processing activities) of the interior and exterior of a building structure, including the surrounding area premises, by an end user or customer of said Kit; whereas, system is comprised of:
a) an instructional media package containing recorded audio-visual media, such as DVD disk and/or VHS tape as well as offering web-based, on-line supplemental information resources, wherein a protocol of step-by-step procedures for conducting said Clandestine Laboratory environmental inspection and for the assessment of the levels of a plurality of predetermined contaminants by the end user or customer of said Kit;
b) an instruction manual defining a protocol of step-by-step procedures for conducting said Clandestine Laboratory environmental inspection and for the assessment of the levels of a plurality of predetermined contaminants by the end user or customer of said Kit;
c) a prepared Kit of tools, materials, and other specialty equipment comprising a reusable shipping cooler container, a quantity of wipe test packs, at least one Vapotrap capsule or canister assembly, two lengths of hose, fittings, an inline particulate filter with hose barbs, a filter element, an AC or DC powered air pump or vacuum unit, a portable timer/thermometer/hygrometer unit, a pre-printed return shipping label, a blue ice packet, at least one pair of disposable latex, nitrile, or rubber gloves, two large rubber bands, an optional diffusive wafer, badge, tube, or cassette, a quantity of additional self sealing plastic bags, an ink pen, a document pack containing a sample log sheet and a test checklist, optional sampling containers, an optional test strip and/or reagent combination pack; and
d) a document pack is comprised of sample log sheets for recording the presence and location of said contaminants and test checklists sheets for recording the findings of the physical inspection activities as well as recording the User's answers to strategic questions that may lend assistance in the interpretation of the analytical results and assist the analytical laboratory in prescribing more precise Kit configurations for follow up inspection activities should they prove necessary.

9. A system and protocol according to claim 1 for providing a clandestine laboratory (Herein termed “Clan-Lab) Home Test Kit for performing the method of claim 1 comprising the steps of:
(a) assembling and providing a Kit of predetermined equipment, materials, and instructional media, which shall consist of both recorded and on-line or web based electronic audio-visual media as well as printed documentation.
(b) providing selected, custom manufactured as well as standard, commercially available, off-the-shelf, testing equipment and materials;
(c) inspecting interior locations within said structure for the presence of contaminants;
(d) inspecting exterior locations upon the premises of said structure as well as the surrounding properties thereto for the presence of contaminants or evidence of clandestine drug lab activity and recording the findings thereof on said checklists;
(e) determining if any suspected contaminants are present within the structure, the type of individual contaminants discovered, and indicate the suspected sampling location thereof as well as the concentration of said contaminants discovered at said location along with a unique identification number or code to allow for said sample to be specifically designated in subsequent analytical results;
(f) recording said sample for a given class of suspected contaminants and the locations thereof on sample log sheet as well as recording the user's answers to strategic assessment related questions on the enclosed checklist forms;
(g) labeling each of said suspected locations with color coded securable labels, stickers, tags or tapes to indicate the type of sampling conducted at said location;
(h) taking samples of a selected number of said suspected class of contaminants from said suspected locations;
(i) containerizing each of said samples within the same container they were originally provided in and preparing said container for shipment;
(j) return shipping of said Clan-Lab Home Test Kit shipping container, including the samples, sampling equipment, and sampling/ checklist documentation therein, to a designated testing laboratory; and
(k) receiving report of results and the explanations thereof from Kit provider or designated laboratory.

10. A process to claim 9 whereas the customer or end user of said Kit chooses the specific test protocol and Kit content via a menu list of selected testing criteria and the associated pricing of each criteria to be provided by Kit provider.

11. The method of claim 9 wherein said checklist and instructional media package provide the user with information and guidance comprising the steps of inspecting the exterior area of said structure as well as the premises thereto and determining the presence and location of suspected contaminants in said exterior area and the areas adjacent thereto.

12. The method according to claim 9 wherein, a plastic or fiber, reusable insulated shipping container is employed for both the sending of the Clan-Lab Home Test Kit media, materials, documentation, and equipment to the customer or end user as well as the user's return shipping of the Kit along with the sampled residues contained therein.

13. The method of claim 9 wherein said checklist comprise forms for said assessment of evidence related to potential contamination originating from illegal clandestine drug manufacturing and processing activities as well as end user recorded information regarding the sampling process and the appropriate return of said samples to a designated analytical laboratory for evaluation.

14. The method of claim 9 wherein said step of taking samples further includes sampling of surfaces for narcotics residues with wipes, swabs, filters, patches, sponges, pads and/or finger cottles, all of which are herein collective referred to as “wipes.”

15. The method of claim 9 wherein the step of taking wipe samples further comprises removal of surface residues using a wipe test apparatus provided in said Kit, and placing said samples in a container provided in said Kit.

16. The method according to claim 9; wherein the wipe sample packs consist of a single prepared wipe unit being place in a color-coded, pre-marked, and sealed smaller bag or other sealable container, which is then placed inside a larger pre-labeled bag along with a pair of disposable gloves for sampling purposes pursuant to the Clan-Lab Home Test Kit process.

17. The method of claim 9 wherein said step of taking samples further includes sampling of interior ambient air concentrations for chemical contaminants including volatile organic compounds (VOC), semi-volatile organic compounds (SVOC), and both organic and inorganic toxic airborne compounds.

18. The method of claim 9 wherein the step of taking samples of said airborne chemical contaminants, including volatile organic compounds (VOC), semi-volatile organic compounds (SVOC), and both organic and inorganic toxic compounds, the collection media and sampling apparatus of which shall be provided in said Kit.

19. The method of claim 9 wherein said step of taking samples further comprises sampling the airborne levels using an electric powered air pump or vacuum unit configured in either an AC or DC voltage version, to force the air flow to be sampled through, upon or drawn through, the adsorption and absorption media collection.

20. The method of claim 9 wherein said step of taking samples further comprises the step of sampling the levels of airborne chemical substances using an air sample collection pump or vacuum unit and placing the sample collection pump or vacuum unit into a pre-marked container provided in said Kit.

21. The method of claim 9 wherein said step of taking samples further comprises the step of sampling the levels of airborne chemical compound concentrations using a self contained air sampling pump or vacuum with a built in or attached media chamber and placing said combination pump/chamber unit containing said sample media in a container provided in said Kit,

22. The method of claim 9 wherein said step of taking samples further comprises the step of static sampling the levels of airborne chemical compound concentrations using diffusive media and placing said air pump or vacuum unit containing said samples in a container provided in said Kit,

23. The method of claim 9 wherein said step of taking samples further comprises sampling of airborne particulates using an air pump or vacuum unit to collect samples via an inline filter assembly as well as the detachment and placement of said filter assembly in a specially marked container provided in said Kit.

24. The method of claim 9 wherein said method further comprises the steps of receiving and evaluating the report of said testing laboratory

25. An apparatus (herein referred to as a Vapotrap Capsule, according to claim 3 wherein is comprised of a capsule containing individually containerized packets or subpods containing certain adsorbent and/or absorbent medias.

26. The device according to claim 3 wherein said capsule and subpods are constructed of a natural or synthetic porous mesh material to allow for unrestricted ventilation within the adsorbent and/or absorbent medias contained therein whereas each mesh packet has a sealing component to:
a) prevent the spillage of the individual adsorbent and/or absorbent media material contained therein,
b) allow for each respective sub-pod to be opened and emptied as may be deemed necessary given the circumstances and facts available relative to the premises being investigated; whereas, an alternative analytical process may become necessary due to the particular challenges presented by the clandestine chemistry considerations by the test location, and
c) allow the subpods to be either emptied or filled by the receiving or supplying laboratory personnel and/or recycled through desorption or the disposal of the same.

27. The method according to claim 3 wherein said subpods are each individually filled with separate types of adsorbent and/or absorbent medias including select grades of activated carbons, zeolites, organic polymers, metal chlorides, silicates, sulfates, silicas, and/or aluminas,

28. The method according to claim 3 wherein certain subpods are selected for the given contamination scenario anticipated, or otherwise indicated as such by previous testing operations, and multiple subpods of adsorbent and/or absorbent materials are placed into the aforedescribed capsule assembly.

29. The method according to claim 3 wherein the capsule assembly containing the aforedescribed sub-capsule packets containing a selected assortment of adsorbent and/or the aforedescribed absorbent media, is placed within the suspect atmosphere to absorb and/or adsorb airborne contaminants through natural indoor convectional airflow processes and to facilitate laboratory analysis of these suspect airborne concentrations of contaminants through the thermal desorption process; wherein said contaminants are common to the practice of clandestine narcotics manufacture and thus include:
(a) organic substances such as volatile organic compounds (VOC), semi-volatile organic compounds (SVOC), and other airborne organic substances such as organic acid or basic vapors; and
(b) inorganic substances including, but not limited to, phosphine, hydrogen chloride, iodine, and ammonia.

30. An apparatus according to claim 25; for containing said Vapotrap Capsule wherein the apparatus (herein referred to as a Vapotrap Canister) consists of a canister body with detachable and sealable end segments as well as valves to facilitate inlet and outlet airflow to said canister assembly as well as a means of attaching said canister assembly to an air-pumping/vacuum device via hose connections or otherwise attaching said air-pumping/vacuum device directly to the said canister assembly. (Reference FIG. 3)

31. The method according to claim 30 wherein the capsule assembly is assembled from metallic components or synthetic plastic and/or fiber substrate materials or a combination of said structural component materials.

32. The method according to claim 30 wherein the aforedescribed capsule assembly is placed within said canister assembly to facilitate the testing of suspect airborne concentrations of contaminants, which are common to the art of clandestine narcotics manufacture including:
(a) organic substances such as volatile organic compounds (VOC), semi-volatile organic compounds (SVOC), and other airborne organic substance such as organic acid or basic vapors; and
(b) inorganic substances such as phosphine, hydrogen chloride, iodine, and ammonia.

33. The method according to claim 30 wherein the canister assembly, fabricated from metallic components, is placed in a heating source unit to facilitate direct analysis of the chemical substances contain within the adsorbent and/or absorbent media components contained therein via the thermal desorption process.

34. The method according to claim 30 wherein the capsule assembly assembled from synthetic plastic and/or fiber substrate materials is opened at the receiving laboratory facility; whereas, the contents of said capsule assembly are emptied from said canister and transferred into a heat resistant canister assembly (made of metallic components) which is then placed in a heating source holster assembly to facilitate direct analysis of the contaminants contained in the adsorbent and/or absorbent components contained therein and thus extract an off-gas flow via the thermal desorption process.

35. The process according to claim 25 wherein said capsule assembly is prepared for laboratory analysis comprising the steps of:
(a) removing said sample from shipping container and cross referencing the attached identification label thereto to the sample log sheet also retrieved from said sample shipping container;
(b) entering the appropriate sample identification code into the customer or end users account file;
(c) removal of said sample from its container packaging;
(d) placing said sample inside an air tight extraction chamber apparatus and sealing the chamber;
(e) introducing a heat and/or steam source to said extraction chamber;
(f) opening a valve located on said extraction chamber once the desired temperature, pressure, and time period are achieved;
(g) routing off-gas flow directly from said extraction chamber into a vapor expansion/mixing tank or routing said off-gas flow indirectly into a vapor expansion/mixing tank via an attached chilling unit assembly designed to reduce the temperature of the off-gas flow as it passes through a length of chilled tube or pipe;
(h) opening a valve on the vapor expansion/mixing tank leading to the analytical instrumentation network;
(i) recording the measurements observed into the end user's account file;
(j) releasing all residual vapors that may be contained said extraction chamber, chilling unit, and/or vapor expansion/mixing tank or the associated piping thereto;
(k) removing sampling media from the extraction chamber and re-sealing the lid assembly; and
(l) purging system with high temperature steam and/or water flow for cleaning prior to introducing another sample for analysis;

36. The process according to claim 30 wherein said canister assembly is prepared for laboratory analysis comprising the steps of:
a) removing said sample canister assembly from shipping container and cross referencing the attached identification label thereto to the sample log sheet also retrieved from said sample shipping container;
b) entering the appropriate sample identification code into the customer or end users account file;
c) placing said sample canister assembly inside a heated yoke assembly for vapor extraction processing;
d) connecting system hoses to said canister assembly and opening both the inlet valve;
e) introducing a heat source to the yoke unit containing the prepared canister assembly;
f) introducing a pressure, heat, and/or steam source to said canister assembly;
g) opening the outlet valve located on said canister assembly once the desired temperature, pressure, and time period are achieved;
h) routing off-gas flow directly from said extraction chamber into a vapor expansion/mixing tank or routing said off-gas flow indirectly into a vapor expansion/mixing tank via an attached chilling unit assembly designed to reduce the temperature of the off-gas flow as it passes through a length of chilled tube or pipe;
i) opening a valve on the vapor expansion/mixing tank leading to the analytical instrumentation network;
j) recording the measurements observed into the end user's account file;
k) releasing all residual vapors that may be contained said canister assembly, chilling unit, and/or vapor expansion/mixing tank or the associated piping thereto;
l) removing canister assembly from the heating yoke unit, empty sampling media capsule from therein and subject said canister assembly to disassembly, cleaning, drying, and reloading with sanitized, prepared media before being redeployed; and
m) purging the remainder of the system with high temperature steam and/or water flow for cleaning prior to processing another sample canister assembly for analysis;

37. A system and protocol according to claim 1, claim 25, and claim 30 for the analytical qualification and quantification of a plurality of gaseous, liquid, and/or solid samples related to the discovery of chemical concentration therein, which may have been associated with past or present illegal clandestine drug manufacturing or processing activities in or upon a structural or real estate premises subject to the testing activities heretofore described in this invention, comprising the steps of:
a. determining by qualification and/or quantification the levels of narcotics residues in wipe samples, swab samples, and/or airborne particulates trapped in or upon filter media by the method of subjecting said residues to analysis via one or more of the following processes including: ion mobility spectrophotometer detection; gas chromatograph surface ionization detection; gas chromatograph mass spectrophotometer detection; Field Ion Spectrophotometer detection; Differential Mobility Spectrometer; Fourier Transform Infrared (or near Infrared) Spectrophotometer detection; Surface Acoustic Wave detection; and Raman Spectrophotometer detection;
b. determining by qualification and/or quantification the levels of organic and inorganic residues in wipe samples, swab samples, and/or airborne particulates trapped in or upon filter media by the method of subjecting said residues to analysis via one or more of the following processes including: gas chromatograph mass spectrophotometer detection; gas chromatograph surface ionization detection; gas chromatograph detection; and Fourier Transform Infrared (or near Infrared) Spectrophotometer detection;
c. determining by qualification and/or quantification the levels of airborne organic and inorganic chemical concentrations in Vapotrap canisters, capsules, and/or subpods by subjecting said concentrations to thermal desorption and analyzing the off-gas flow via one or more of the following processes including: gas chromatograph mass spectrophotometer detection; gas chromatograph surface ionization detection; gas chromatograph detection; and Fourier Transform Infrared (or near Infrared) Spectrophotometer detection;
d. determining by qualification and/or quantification the levels of airborne organic and inorganic chemical concentrations in adsorbent media in the forms of diffusive wafers, badges, cassettes, disks, tubes, and/or cartridges, by subjecting said medias to thermal desorption and analyzing the off-gas flow via one or more of the following processes including: gas chromatograph mass spectrophotometer detection; gas chromatograph surface ionization detection; gas chromatograph detection; and Fourier Transform Infrared (or near Infrared) Spectrophotometer detection;
e. determining by qualification and/or quantification the levels of liquid-based organic and inorganic chemical concentrations in surface and groundwater samples by subjecting said samples to thermal desorption and analyzing the headspace off-gas flow via one or more of the following processes including: gas chromatograph mass spectrophotometer detection; gas chromatograph surface ionization detection; gas chromatograph detection; and Fourier Transform Infrared (or near Infrared) Spectrophotometer detection;
f. determining by qualification and/or quantification the levels of organic and inorganic chemical concentrations in soil and suspect solid samples by subjecting said samples to thermal desorption and analyzing the headspace off-gas flow via one or more of the following processes including: gas chromatograph mass spectrophotometer detection; gas chromatograph surface ionization detection; gas chromatograph detection; and Fourier Transform Infrared (or near Infrared) Spectrophotometer detection;
g. determining by qualification and/or quantification the levels of organic and inorganic chemical concentrations in soil and suspect solid samples by subjecting said samples to infrared, and near infrared, spectrum analysis via the non-destructive spectrophotometer detection process, such as is offered by the Raman Spectrophotometer and Ion Mobility Spectrophotometer devices.
h. determining or qualifying the suspected presence of organic and inorganic chemical concentrations in soil and suspect solid samples by subjecting said samples to direct screening via the processes of photoionization detection and/or flame ionization detection; and
i. determining or qualifying the suspected presence of organic and inorganic chemical concentrations in water and suspect liquid samples by subjecting said samples to direct screening via the processes of photoionization detection and/or flame ionization detection;

38. A method according to claim 9; wherein, a spectrophotometer is used for sampling leachate extracted from inorganic wipe sample liquid residues as a means of confirming inorganic metal film presence on suspect surface areas associated with an investigated structure.

39. A method of claim 37 wherein comprising the detection and identification of analytes in a sample, comprising: (a) the thermal desorption of said analytes via an introduced heat or steam source volatilizing at least a portion of the sample to produce a volatilized sample that includes analyte contaminants and may also include doplant gas or markers detectable by an aspect of spectrophotometry or other analytical measurement instrumentation according to the processes heretofore described, thereby detecting and identifying at least one analyte in the sample.

40. A method of claim 37 wherein said method further comprises the step of generating a report on the results of said environmental assessment of said property and either physically or electronically deliver and/or communicate said results to the user of the Clandestine Laboratory Home Test Kit.

41. A system and protocol for claim 1 wherein the Clandestine Laboratory (Clan-Lab) Home Test Kit is arranged in three basic configurations as per the end user's specification and these configurations are as follows:

| | |
|---|---|
| a. Clan-Lab Home Test Kit - Q1 | (Qualitative) |
| Configuration | |
| b. Clan-Lab Home Test Kit - Q2 | (Quantitative) |
| Configuration | |
| c. Clan-Lab Home Test Kit - Q3 | (Combination - A specified |
| Configuration | combination of Qualitative (Q1) |
| | and Quantitative (Q2) |
| | Configuration Components or |
| | optional components as per the |
| | end user or customer's |
| | specification.) |

42. A system and protocol according to claim 1 for providing a Clan-Lab Home Test Kit in a Custom Combination Configuration (herein referred to as Q3 configuration) comprising one or more of the following steps of:
a) taking samples of said airborne chemical substances comprises the step of collecting said samples in an Vapotrap Capsule or Canister Assembly or other forms of commercially available adsorbent media described herein and placing said sample in a pre-marked, designated container provided in said Kit;
b) taking samples of narcotics residue and subjecting said samples to the tests included in the Colorimetric Reagent Test Pack to identify narcotic substance residues as well as and their precursor and recursor products and byproducts;
c) taking samples of suspect residues and subjecting said samples to the tests included in the Colorimetric Test Strip Pack to identify illegal drug manufacturing byproducts and waste material residues which is useful for forensically determining the method of drug manufacture and the anticipated chemistry utilized in the clandestine laboratory operation;
d) including a calorimetric iodine marker test, which is provided to confirm the red phosphorus (or “Red P”) method of illegal methamphetamine manufacture; wherein, a wipe pack, pre-saturated with liquid starch, is wiped across a suspect surface for the purpose of identifying the presence of iodine residues on said suspect surfaces as would be positively confirmed by a purple color change occurring immediately in the saturated wipe upon contact with the residues.
e) taking swab samples of narcotics residue from within otherwise inaccessible locations such as light fixtures, HVAC systems, and appliance vents pursuant to the Clan-Lab Home Test Kit process;
f) taking samples of said surface water further comprises collecting said surface water in a sterile jar provided in said Kit;
g) including a pre-labeled, sanitized glass or plastic container, a sanitized sealable lid, and a sanitized hand scoop tool in said Kit configuration as a method of allowing soil sampling to be conducted in instances where the end user has observed what may be burn pile residue, a chemical dumping area, or other evidence pursuant to suspected illegal drug manufacturing activity;
h) including a pre-labeled, sanitized glass or plastic container and a sanitized, sealable lid as well as a bailing device with an attached length of cord in said Kit configuration as a method of allowing surface water, groundwater, and/or septic tank fluids to be sampled in instances where the end user has reason to suspect that such fluids may have been contaminated pursuant to illegal drug manufacturing activity; and
i) including, as a means of analytical comparison measurement, commercially available airborne chemical detection tubes, including tubes such as those manufactured by Draeger, Gastec, Sensidyne, Rae, Kitagawa, Tenax, or MSA as well as the associated pump or vacuum unit apparatus thereof and non-vented or static diffusion tubes; and commercially available adsorbent media in the forms of diffusive wafers, badges, cassettes, disks, tubes, and/or cartridges.

43. The method according to claim 1 whereas the commercially available airborne chemical detection tubes, including tubes such as those manufactured by Draeger, Gastec, Sensidyne, Rae, Kitagawa, Tenax, or MSA as well as the associated pump or vacuum unit apparatus thereof and non-vented or static diffusion tubes, are incorporated as a component/s within a more comprehensive Clandestine Laboratory Home Test Kit configuration for the purpose of allowing regular citizens of average intelligence or end users a means of self-determining if there exists evidence of toxic chemical concentrations pursuant to an investigation of a suspect premises that may have been subject to impact by past or present activities associated with clandestine drug manufacturing or processing.

44. The method according to claim 1 of using of commercially available adsorbent media in the forms of diffusive wafers, badges, cassettes, disks, tubes, and/or cartridges, which were originally developed as devices to be worn by workers for the purposes of measuring of workplace related hazardous chemical exposures; wherein the improvement comprises using these adsorbent media as a component in a comprehensive Clandestine Laboratory Home Test Kit, containing a variety of diverse testing apparatus, for the purpose of determining the identity and quantity of airborne chemical concentrations pursuant to a given area atmosphere within a structure for evidence of toxic hazards associated with illegal drug manufacturing and/or processing activities.

45. A process for claim 41 whereas the Clandestine Laboratory (Clan-Lab) Home Test Kit's Q3 (or Combination Configuration), which consists of user selected components from the Q1 or Qualitative Configuration and the Q2 or Quantitative Configuration as well as selected diagnostic components or test packs, is employed by the end user or customer as a follow-up measure to a previous testing session; wherein the qualitative and/or quantitative testing of the subject premises indicated the presence of chemical substances and justified further investigation activities; in that, the Q3 Configuration will offer the user a very specific arrangement of testing protocol in order to:
a) further identify and quantify chemicals of concern;
b) to discover the physical distribution or area impacted by said chemicals;
c) to be able to delineate the magnitude and distribution of the toxic hazard impact said chemicals substances have had upon of the subject premises;
d) to be able to make intelligent, responsible, timely decisions about premises occupancy situations thereby presenting a risk to human life and health;
e) be able to estimate the cost of repair and remediation of the damages sustained upon said premises by the clandestine drug manufacturing or processing acts; and
f) allow the end user to have an accurate objective assessment of said damages as well a means of determining the costs required to restore said premises; in that, the user may seek relief and restitution from insurance carriers, victim assistance fund resources, and/or judicial relief pursuant to court ordered restitution actions.

46. A method according to claim 41 whereas the Clandestine Laboratory (Clan-Lab) Home Test Kit's Q3 (or Combination Configuration), includes one or more the following user selected diagnostic components or Colorimetric Reagent Test Pack to determine the identity, distribution, and damage of a designated chemical substance upon a subject property; whereas the optional test pack Kit arrangements claimed in this said test pack system are comprised of:
a) Simon's reagent for indicating secondary amines such as 3,4-methylenedioxymeth-amphetamine (MDMA), methylenedioxyamphetamine (MDA) derivatives, or Methamphetamine;
b) Robadope reagent for indicating primary amines such as methylenedioxyamphetamine (MDA), paramethoxyamphetamine (PMA), or Amphetamine;
c) Mecke reagent for Ecstasy, ‘XTC’, DXM and substances from the 2-CT-XX family;
d) Dille-Koppanyi reagent for identifying barbiturates;
e) Duquenois-Levine reagent for marijuana/tetrahydrocannibinol;
f) Cobalt isothiocyanate reagent for cocaine;
g) Ehrlich's Modified reagent for LSD;
h) Nitnc Acid reagent for morphine and heroin compounds;
i) Mayer's reagent for narcotic alkaloids;
j) Silver/Copper reagent for Gamma Hydroxybutyrate (GHB) analogs and compounds;
k) Chen's reagent for ephedrine and pseudoephedrine compounds; and
l) The general indicator component pack of Marquis reagent, Mandelin reagent, and Liebennann's reagent assortment, which tests not only indicate morphine, opiates, and hydrochlorides, but also indicates a large variety of other controlled substance narcotics materials according to a specific given color arrangement.

47. A method according to claim 41 whereas the Clandestine Laboratory (Clan-Lab) Home Test Kit's Q3 (or Combination Configuration), includes one or more the following user selected diagnostic components or Colorimetric Test Strip Pack to determine the identity, distribution, and damage of certain designated narcotics precursor and recursor chemical substances upon a subject property; whereas the optional test pack Kit arrangements claimed in this said test pack system are comprised of:
a) pH test strips are as a method of colorimetrically determining the presence and location of potential corrosive film deposits on suspect surface areas pursuant to the Clan-Lab Home Test Kit process;
b) methyl Yellow test paper/strips are included in said Kit configuration for confirming the presence of significant ammonia/ ammonium compound residues; whereas, these test strips are a method of calorimetrically determining the presence and location of anhydrous ammonia residue film deposits on suspect surface areas relative to the ammonia or “Nazi” method of clandestine drug manufacture;
c) Molybdic acid test strips are included in said Kit configuration as method of calorimetrically determining the presence and location of phosphorous, phosphine, and/or phosgene residue film deposits on suspect surface areas pursuant to the Clan-Lab Home Test Kit process;
d) Phosphate test strips are included in said Kit configuration as a method of calorimetrically determining the presence and location of phosphorous, phosphine, and/or phosgene residue film deposits on suspect surface areas relative to the Red Phosphorus or “Red P” method of clandestine drug manufacture;
e) Lithium test strips/paper are included in said Kit configuration as a method of calorimetrically determining the presence and location of as a method of measuring lithium residues as a marker or precursor chemical for forensically qualifying the chemistry employed in particular illegal drug manufacturing practices and are relative to the Birch or “Nazi” method of clandestine drug manufacture;
f) Lead test strips and Mercury test strips are included in said Kit configuration as a method of calorimetrically determining the presence and location of lead and/or mercury residue film deposits on suspect surface areas relative to the phenyl-2-propanone (P2P) or “Amalgam” method of illegal clandestine drug manufacture;

48. A method according to claim 41 whereas the Clandestine Laboratory (Clan-Lab) Home Test Kit's Q3 (or Combination Configuration), includes one or more the following user selected diagnostic components or Suspect Materials Test Pack to determine the identity, distribution, and damage that narcotics related compounds or chemical substances may have had by means of contact and/or absorption upon an item to be sampled from a structure's composition or contents; whereas the optional test pack Kit arrangements claimed in this said test pack system are comprised of:
a) a boring or coring instrument for cutting, boring, or sawing a sample of wallboard, drywall, plaster, or other article of a structure's interior such as wall, ceiling, and/or floor materials;
b) a cutting instrument such as a knife or other bladed instrument or a saw for extracting a sample from: an object of structural media: an object of furnishing; an object of floor, wall, or window covering; an object of personal or business property; or other object from a structure's composition or contents;
c) a pre-marked, color-coded sample container;
d) a pre-marked, container for the supply and return of the enclosed sampling tools to the Kit provider;
e) an instructional media package, which illustrates and defines the unique considerations and details involved in this sampling process; and
f) a structural materials sample log sheet for collecting information from the user of said Kit about the sampling process as well as defining the arrangement, area, and scope of the substrate or object being sampled and its relationship to the structure.

49. A method according to claim 41 whereas the Clandestine Laboratory (Clan-Lab) Home Test Kit's Q3 (or Combination Configuration), includes a Photoionization Meter Pack to provide a Kit user with a pre-calibrated, pre-tested electronic photoionization instrument on a rental or usage only basis for a given period of time so that the user may use said instrument to determine the presence, distribution, and damage that narcotics related compounds or chemical substances may have had upon objects of a structure's composition or contents; whereas the optional test pack Kit arrangements claimed in this said test pack system are comprised of:
a) an electronic photoionization meter;
b) a photoionization meter calibration apparatus;
c) an instructional media package, which illustrates and defines the proper operation of the photoionization meter and is comprised of details of said meter investigation process; and
d) a container for said meter pack.

50. A method, system and protocol according to claim 25 and claim 30, wherein said devices and systems are used for analytical qualification and quantification of a plurality of airborne or gaseous samples related to the discovery of chemical concentrations, which are therein related to all industrial hygiene, environmental, and health and safety applications wherein said apparatus is used for determining the identity and quantity of organic, inorganic, and/or biological contaminants.

51. A method, system and protocol according to claim 30, wherein said device is configured with an in-line particulates filter assembly and removable filter element.

52. A method according to claim 21, wherein a device is configured to include an air-pumping or vacuum mechanism, configured in either an AC or DC voltage version, to force the air flow to be sampled through, upon or drawn through an accessible and sealable chamber for holding an adsorption media, or collection of said medias;
whereas said device is equipped with an electronic microprocessor and sensor array to monitor and record information about the testing operation process including the time, date, temperature and relative humidity variation and record such data onto a storage media thus allowing subsequent downloading of said information by the Kit provider or receiving laboratory unit to be used and factored into the analysis record of the sampling event.
